(19) United States
(12) Patent Application Publication
(10) Pub. No.: US 2021/0069257 A1
(43) Pub. Date: Mar. 11, 2021

(54) COMPOSITION AND METHOD FOR TREATING SKIN CONDITION

(71) Applicant: PREDICTIVE BIOTECH, INC., Sait Lake City, UT (US)

(72) Inventors: Eric K. OLSON, Salt Lake City, UT (US); Bradley C. ROBINSON, Salt Lake City, UT (US)

(21) Appl. No.: 16/639,353

(22) PCT Filed: Aug. 15, 2018
(86) PCT No.: PCT/US2018/000194

§ 371 Date: Feb. 14, 2020

Related U.S. Application Data
(60) Provisional application No. 62/545,750, filed on Aug. 15, 2017.

(51) Int. Cl. A61K 35/51; A61K 35/28; A61K 35/50; A61P 17/02; A61K 47/36; A61K 47/10; A61K 9/06

(52) U.S. Cl. CPC A61K 35/51; A61K 35/28; A61K 35/50; A61K 9/06; A61K 47/36; A61K 47/10; A61P 17/02

(57) ABSTRACT

Disclosed herein are compositions, formulations, methods, and kits for treating a skin condition.

### BACKGROUND OF THE INVENTION

Skin conditions, such as wounds, can be devastating to patients. There are an estimated 500,000 burns treated in the United States each year and globally, this statistic increases to 11 million injuries per year. The overall mortality rate for burn injury was 4.9% between 1998-2007 and medical costs for burn treatments approach $2 billion per year. In addition, chronic wounds constitute a large patient base and healing rates remain below 50%. These non-healing chronic wounds are estimated to effect 7 million people per year in the United States, with yearly costs approaching $25 billion.

Patients who suffer from these skin conditions can benefit from rapid treatments that result in at least partial closure and protection of the wounds. Disclosed herein are compositions, formulations, methods, and kits for treating the skin conditions.

### SUMMARY OF THE INVENTION

Disclosed herein are compositions, formulations, methods, and kits for treating a skin condition.

In one aspect, disclosed is a composition comprising: (a) an isolated and processed birthing tissue comprising an umbilical cord tissue, wherein said birthing tissue comprises from about 10% to about 95% by weight of said composition, and (b) at least one of: (i) a plurality of isolated stem cells, (ii) an isolated inductive component comprising a portion of a Wharton's jelly, (iii) a viscosity modifying component, and (iv) a cryopreservative. In some embodiments, the composition comprises the viscosity modifying component. In some embodiments, the viscosity modifying component comprises a polyol, a sugar, a derivative of any of these, a salt of any of these, or any combination thereof. In some embodiments, the viscosity modifying component comprises a polyol, a derivative thereof, a salt of any of these, or any combination thereof. In some cases, the birthing tissue comprises from about 10% to about 95% by weight of the composition, for example, about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 95%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 95%, about 20% to about 25%, about 20% to about 30%, about 20% to about 40%, about 20% to about 50%, about 20% to about 60%, about 20% to about 70%, about 20% to about 80%, about 20% to about 90%, about 20% to about 95%, about 25% to about 30%, about 25% to about 40%, about 25% to about 50%, about 25% to about 60%, about 25% to about 70%, about 25% to about 80%, about 25% to about 90%, about 25% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% by weight of the composition.

In some embodiments, the polyol, the derivative thereof, or the salt of any of these is a polyhydric alcohol, a derivative thereof, or a salt of any of these. In some embodiments, the polyhydric alcohol, the derivative thereof, or the salt of any of these is a glycerol, an erythritol, a threitol, an arabitol, a xylitol, a ribitol, a mannitol, a sorbitol, a galactitol, a fucitol, an iditol, an inositol, a volemitol, an isomalt, a maltitol, a lactitol, a maltotriitol, a maltotetraitol, a polyglycitol, a salt of any of these, a derivative of any of these, or any combination thereof. In some embodiments, the salt of any of these or the derivative of any of these, and wherein the salt of any of these or the derivative of any of these is alkylated, acetylated, phosphorylated, ubiquitinated, glycosylated, methylated, hydroxylated, sulfated, sulfonated, pegylated, propylene glycolated, contains an amino group, contains an imino group, or any combination thereof. In some embodiments, the polyhydric alcohol or the salt thereof is a C₃-C₃₀ polyhydric alcohol, a derivative thereof, or a salt of any of these.

In some embodiments, the viscosity modifying component comprises a polymer. In some embodiments, the polymer comprises a plurality of sugar monomers. In some embodiments, the polymer comprises an alginic acid or a salt thereof. In some embodiments, the viscosity modifying component comprises a carboxylic acid or a salt thereof. In some embodiments, the viscosity modifying component comprises a gelatin or a derivative thereof.

In some embodiments, the viscosity modifying component comprises collagen. In some embodiments, the collagen is at least partially hydrolyzed.

In some embodiments, the viscosity modifying component is selected from the group consisting of: carboxylic acid copolymers, carboxylate copolymers, acrylate homopolymers, acrylate copolymers, and any combination thereof.

In some embodiments, the viscosity modifying component is from about 1% to about 20% by weight of said composition. In some embodiments, the viscosity modifying component is from about 12% to about 17% by weight of said composition.

In some embodiments, the composition comprises the plurality of isolated stem cells. In some embodiments, the plurality of isolated stem cells comprises mesenchymal stem cells (MSCs). In some embodiments, the plurality of isolated stem cells comprises an autologous stem cell. In some embodiments, the plurality of isolated stem cells comprises a heterologous stem cell. In some embodiments, at least a portion of the plurality of stem cells is viable. In some embodiments, the at least a portion is at least about 80% of a total cell number of the composition. In some embodiments, the plurality of isolated stem cells is from about 0.1% to about 5% by weight of said composition. In some embodiments, the plurality of isolated stem cells is from about 0.5% to about 2% by weight of said composition.

In some embodiments, the composition comprises the isolated inductive component. In some embodiments, the isolated inductive component comprises a mucous tissue. In some embodiments, the isolated inductive component comprises a mucopolysaccharide or a salt thereof. In some embodiments, the isolated inductive component comprises hyaluronic acid or a salt thereof. In some embodiments, the isolated inductive component comprises chondroitin or a salt thereof. In some embodiments, the isolated inductive component comprises a salt of chondroitin. In some embodiments, the isolated inductive component comprises N-acetylgalactoasmine or a salt thereof. In some embodiments, the isolated inductive component comprises glucuronic acid or a salt thereof. In some embodiments, the isolated inductive component comprises water. In some embodiments, the isolated inductive component comprises a plurality of isolated fibroblasts. In some embodiments, at least a portion of the plurality of isolated fibroblasts is viable. In some embodiments, the isolated inductive component comprises a plurality of isolated macrophages. In some embodiments, at least a portion of the plurality of isolated macrophages is viable.

In some embodiments, the isolated inductive component is from about 0.1% to about 50% by weight of said composition. In some embodiments, the isolated inductive component is from about 30% to about 50% by weight of the composition.

In some embodiments, the composition comprises the isolated and processed birthing tissue. In some embodiments, the isolated and processed birthing tissue further comprises a placental tissue. In some embodiments, the isolated and processed birthing tissue comprises a conductive property. In some embodiments, the isolated and processed birthing tissue comprises minced tissue. In some embodiments, the isolated and processed birthing tissue comprises micronized tissue. In some embodiments, the isolated and processed birthing tissue lyophilized tissue. In some embodiments, the isolated and processed birthing tissue comprises a plurality of pieces of tissue.

In some embodiments, the isolated and processed birthing tissue is from about 20% to about 50% by weight of the composition. In some embodiments, the isolated and processed birthing tissue is from about 30% to about 50% by weight of the composition.

In some embodiments, the composition further comprises a plurality of epithelial cells. In some embodiments, the composition further comprises a plurality of endothelial cells. In some embodiments, the composition further comprises a plurality of perivascular cells. In some embodiments, the composition further comprises a peptide. In some embodiments, the composition further comprises a protein. In some embodiments, the composition further comprises an amino acid. In some embodiments, the composition further comprises water.

In some embodiments, the composition further comprises at least one growth factor. In some embodiments, the at least one growth factor comprises insulin-like growth factor-1, insulin-like growth factor binding protein-3, vascular endothelial growth factor (VEGF), hepatocyte growth factor (HGF), placenta growth factor (PLGF), or any combination thereof.

In some embodiments, the composition further comprises at least one of: chemokine ligand 2, macrophage inflammatory protein-1 (MIP-1) alpha, MIP-1 beta, MIP-2, beta-chemokine ligand-5, beta-chemokine ligand-20, alpha-chemokine ligand-14, lipopolysaccharide-induced alpha-chemokine, Granulocyte-macrophage colony-stimulating factor, interleukin IL-1 beta, phorbol myristate acetate, epidermal growth factor, fibroblast growth factor, vascular endothelial growth factor, connective tissue growth factor, platelet-derived growth factor, insulin-like growth factor, nerve growth factor, hepatocyte growth factor, colony-stimulating factor, stem cell factor, keratinocyte growth factor, granulocyte colony-stimulating factor, granulocyte macrophage colony-stimulating factor, glial derived neurotrophic factor, ciliary neurotrophic factor, endothelial-monocyte activating polypeptide, epithelial neutrophil activating peptide, erythropoietin, bone morphogenetic protein, brain-derived neurotrophic factor, transforming growth factor beta, tumor necrosis factor, or any combination thereof.

In some embodiments, the composition further comprises at least one hormone. In some embodiments, the at least one hormone is prolactin or leptin.

In some embodiments, the composition comprises the cryopreservative. In some embodiments, the cryopreservative comprises a sucrose, a trehalose, a starch, a salt of any of these, a derivative of any of these, or any combination thereof.

In some embodiments, the composition is in unit dose form. In some embodiments, the composition is formulated as a liquid, an aerosol, an aerosolized liquid, a foam, a cream, a gel, an ointment, a putty, a semi-solid, a glue, a coating, an adhesive, or any combination thereof.

In some embodiments, the composition comprises a viscosity from about 0.5 centipoise (cp) to about 2 cp at 70° F. In some embodiments, the composition comprises a viscosity from about 100 cp to about 900 cp at 70° F. In some embodiments, the composition comprises a viscosity from about 1,000 cp to about 5,000 cp at 70° F. In some embodiments, the composition comprises a viscosity from about 50,000 cp to about 80,000 cp at 70° F. In some embodiments, the composition comprises a viscosity from about 90,000 cp to about 150,000 cp at 70° F. In some embodiments, the composition comprises a viscosity from about 200,000 cp to about 300,000 cp at 70° F.

In some embodiments, the composition maintains at least about 90% of a wound healing activity after storage in a container at an average temperature from about 25 degrees Celsius to about 200 degrees Celsius. In some embodiments, the container is a sealed container.

In another aspect, the disclosed herein is a method comprising: administering the composition to a subject. In some embodiments, the subject is a subject in need thereof. In some embodiments, the subject is a human subject. In some embodiments, the subject is a subject having or suspected of having a condition. In some embodiments, the condition comprises a skin condition.

In some embodiments, the time of healing from the condition for the subject is reduced by at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, when compared to another time of healing for a comparable condition for another subject, wherein the comparable condition of the another subject is not treated with the composition.

In some embodiments, the administering comprises contacting the composition to an abrasion, an incision, a peel, a laceration, a puncture, an avulsion, a contusion, an amputation, a chemical wound, a thermal wound, an electrical wound, an ulcer, a sore, a hematoma, a burn, or any combination thereof.

In another aspect, the disclosed herein is a process comprising: forming a composition by combining (a) an isolated and processed birthing tissue comprising an umbilical cord tissue, wherein said birthing tissue comprises from about 10% to about 95% by weight of said composition, and (b) at least one of: (i) a plurality of isolated stem cells, (ii) an isolated inductive component comprising a portion of a Wharton's jelly, (iii) a viscosity modifying component, and (iv) a cryopreservative. In some cases, the birthing tissue comprises from about 10% to about 95% by weight of the composition, for example, about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 10% to about 95%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 15% to about 95%, about 20% to about 25%, about 20% to about 30%, about 20% to about 40%, about 20% to about 50%, about 20% to about 60%, about 20% to about 70%, about 20% to about 80%, about 20% to about 90%, about 20% to about 95%, about 25% to about 30%, about 25% to about 40%, about 25% to about 50%, about 25% to about 60%, about 25% to about 70%, about 25% to about 80%, about 25% to about 90%, about 25% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95% by weight of the composition.

In some embodiments, the process further comprises: adding a preservative, an anti-irritant, or a combination thereof to the composition.

In some embodiments, the process further comprises: treating the composition with a solution comprising at least one selected from the group consisting of: an antibiotic, an antimycotic, and a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Disclosed herein are compositions, formulations, methods, and kits for treating a skin condition. In some cases, the skin condition is a wound. In some cases, the wound is an injury to living tissue. In some cases, the wound is an acute wound. In some cases, the wound is a chronic wound. In some cases, the wound is caused by penetrating trauma. In some cases, the wound is caused by non-penetrating trauma. In some cases, the wound is a surgical wound. In some cases, the wound is a cut or incision made during a surgery. In some cases, the wound is a cut or incision made during a transplant surgery. In some cases, the wound is a cut or incision made by a scalpel. In some cases, the wound is caused by a medical illness, such as diabetes, atherosclerosis, or deep vein thrombosis. In some cases, the wound is a diabetic wound. For example, the wounds can be treated by the compositions, formulations, methods, and kits disclosed herein include, but are not limited to, abrasions, avulsions, blowing wounds, burn wounds, contusions, gunshot wounds, incised wounds, open wounds, penetrating wounds, perforating wounds, puncture wounds, seton wounds, stab wounds, surgical wounds, subcutaneous wounds, and tangential wounds.

### Definitions

The terminology used herein is for the purpose of describing particular cases only and is not intended to be limiting. As used herein, the singular forms “a”, “an” and “the” can be intended to include the plural forms as well, unless the context clearly indicates otherwise. Furthermore, to the extent that the terms “including”, “includes”, “having”, “has”, “with”, or variants thereof can be used in either the detailed description and/or the claims, such terms can be intended to be inclusive in a manner similar to the term “comprising”.

The term “skin defect” as used herein can refer to any dermatological disorder such as a wound, eruption, skin cancer, rash, burn, tumor, ulcer, boil, irritation, incision, cut, and trauma. The term “skin defect” can be used interchangeably with any of these terms throughout the specification, depending upon the context in which the term is used.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values may be described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. For example, an active agent that is “substantially localized” in an organ can indicate that about 90% by weight of an active agent, salt, or metabolite can be present in an organ relative to a total amount of an active agent, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “subject”, “patient” or “individual” as used herein can encompass a mammal and a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-18 years old. In some instances, a human can be age 18-130 years. In some instances, a subject can be a female. In some instances, a subject can be diagnosed with, or can be suspected of having, a condition or disease. A subject can be a patient. A subject can be an individual. In some instances, a subject, patient or individual can be used interchangeably.

The term “preventing” can mean preventing additional symptoms, ameliorating or preventing the underlying metabolic causes of symptoms, and can include prophylaxis.

In some instances, “treat,” “treating”, “treatment,” “ameliorate” or “ameliorating” and other grammatical equivalents can include prophylaxis. “Treat,” “treating”, “treatment,” “ameliorate” or “ameliorating” and other grammatical equivalents can further include achieving a therapeutic benefit and/or a prophylactic benefit. Therapeutic benefit can mean eradication of the underlying disease being treated. Also, a therapeutic benefit can be achieved with the eradication of one or more of the physiological symptoms associated with the underlying disease such that an improvement can be observed in a subject notwithstanding that, in some embodiments, the subject can still be afflicted with the underlying disease.

The terms “effective amount”, “therapeutically effective amount” or “pharmaceutically effective amount” as used herein, can refer to a sufficient amount of a compound being administered which will at least partially ameliorate a symptom of a disease or condition being treated.

The terms “compound”, “agent”, “active agent”, or active ingredient can be used to refer to a drug or therapeutic as described herein. In some cases, the terms “additional compound”, “additional agent”, or “additional therapeutic agent” can be used interchangeably to refer to other active compounds, agents, or therapeutics that may be used in a composition described herein.

The terms “administer,” “administering”, “administration,” and the like, as used herein, can refer to methods that can be used to enable delivery of compounds or compositions to the desired site of biological action. These methods can include oral administration, intraduodenal administration, parenteral administration (including intravenous, subcutaneous, intrathecal, intraperitoneal, intramuscular, intravascular or infusion), topical and rectal administration. In some instances, a subject can administer the composition in the absence of supervision. In some instances, a subject can administer the composition under the supervision of a medical professional (e.g., a physician, nurse, physician's assistant, orderly, hospice worker, etc.).

The terms “pharmaceutically acceptable salt” or simply “salt” as used herein, can refer to a salt that retains at least some of the biological effectiveness of the free acids and bases of the specified compound. In some instances, the salt can be not biologically or otherwise undesirable. In some embodiments, a compound disclosed herein can possess acidic or basic groups and therefore can react with any of a number of inorganic or organic bases, and inorganic and organic acids, to form a pharmaceutically acceptable salt. In some embodiments, a salt can be prepared in situ during the final isolation and purification of a compound, or by separately reacting a purified compound in its free base form with a suitable organic or inorganic acid, and isolating the salt thus formed.

Examples of pharmaceutically acceptable salts can include those salts prepared by reaction of a compound disclosed herein with a mineral, organic acid or inorganic base, such salts can include, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate and xylenesulfonate.

The terms “co-administration”, “administered in combination with” and their grammatical equivalents or the like, as used herein, can encompass administration of selected therapeutic agents to a single patient, and can include treatment regimens in which the agents can be administered by the same or different route of administration or at the same or different times. In some embodiments, a compound disclosed herein can be co-administered with other agents. These terms can encompass administration of two or more agents to an animal so that both agents and/or their metabolites can be present in the animal at the same time. They can include simultaneous administration in separate compositions, administration at different times in separate compositions, and/or administration in a composition in which both agents can be present. Thus, in some embodiments, a compound and another agent(s) can be administered in a single composition. In some embodiments, a compound and another agent(s) can be admixed in the composition.

The term “Good Wound Care (GWC)” as used herein can refer to the steps or procedures to take care of a wound. For example, good wound care practices can include, but are not limited to, one or more of the following: debridement (e.g., surgical/sharp, mechanical, autolytic or chemical/enzymatic), cleaning (e.g., routine wound cleansing with, e.g., saline), dressings, pressure relief (e.g., off-loading pressure to the foot), maintenance of moist wound environment, and/or infection control (e.g., antibiotic ointment or pills). Optionally, good wound care practice can include fitting a subject with comfortable, cushioned footwear, nutritional support, maintaining blood glucose control, management of other risk factors (e.g., weight, smoking), etc. Good wound care can include one or more of the above practices.

The term “autologous” as used herein can refer to a transplant process in which the donor and recipient of the plurality of isolated stem cells are the same individual. Thus, in an autologous transplant process stem cells can be harvested from a subject and then returned to the same subject. In contrast, “heterologous” as used herein can refer to a transplant process in which the donor and recipient of the plurality of isolated stem cells are not the same subject.

The term “angiogenic factor or agent” as used herein can refer to a growth factor or its receptor which is involved in stimulating the development of blood vessels, e.g., promote angiogenesis, endothelial cell growth, stability of blood vessels, and/or vasculogenesis, etc. For example, angiogenic factors, can include, but are not limited to, e.g., VEGF and members of the VEGF family and their receptors (VEGF-B, VEGF-C, VEGF-D, VEGFR1, VEGFR2 and VEGFR3), P1GF, PDGF family, fibroblast growth factor family (FGFs), TIE ligands (Angiopoietins, ANGPT1, ANGPT2), TIE1, TIE2, ephrins, Bv8, Delta-like ligand 4 (DLL4), Del-I, fibroblast growth factors: acidic (aFGF) and basic (bFGF), FGF4, FGF9, BMP9, BMP1O, Follistatin, Granulocyte colony-stimulating factor (G-CSF), GM-CSF, Hepatocyte growth factor (HGF)/scatter factor (SF), Interleukin-8 (IL-8), CXCL12, Leptin, Midkine, neuropilins, NRP1, NRP2, Placental growth factor, Platelet-derived endothelial cell growth factor (PD-ECGF), Platelet-derived growth factor, especially PDGF-BB, PDGFR-alpha, or PDGFR-beta, Pleiotrophin (PTN), Progranulin, Proliferin, Transforming growth factor-alpha (TGF-alpha), Transforming growth factor-beta (TGF-beta), Tumor necrosis factor-alpha (TNF-alpha), AIk 1, CXCR4, Notch1, Notch4, Sema3A, Sema3C, Sema3F, Robo4, etc. Angiogenic factor or agent can further include factors that promote angiogenesis, such as ESM1 and Perlecan. It can also include factors that accelerate wound healing, such as growth hormone, insulin-like growth factor-I (IGF-I), VIGF, epidermal growth factor (EGF), EGF-like domain, multiple 7 (EGFL7), connective tissue growth factor (CTGF) and members of its family, and TGF-alpha and TGF-beta.

The term “differentiation” as used herein can refer to the process of cell development with an increase in the level of organization or complexity of a cell or tissue, accompanied with a more specialized function.

The terms “dose” and “dosage” as used herein can be used interchangeably to refer to an amount of an active agent or a pharmaceutical composition administered to a subject.

The term “chronic wound” as used herein can refer to a wound that does not heal. An acute wound can develop into a chronic wound. Acute wounds can include, but are not limited to, wounds caused by, e.g., thermal injury, trauma, surgery, excision of extensive skin cancer, deep fungal and bacterial infections, vasculitis, scleroderma, pemphigus, toxic epidermal necrolysis, etc. The term “normal wound” as used herein can refer to a wound that undergoes normal wound healing repair. Subjects treated with the composition disclosed herein can have at least one wound. The wound can be a chronic, acute or normal wound.

### Wound Healing

Skin can provide a shield against harmful substances and organisms such as bacteria and parasites. It can help to keep moisture within the body and keep one's body temperature constant. It can also help one feel sensations to external stimulus. Upon an injury to the skin, the tissues underneath the skin can be exposed to infection and other complications. Therefore, effective healing is critical to the reconstruction of new skin as a barrier. However, wound healing can be a complex process involving inflammation, granulation tissue formation, reepithelization and remodeling. At the site of the wound there can be many processes occurring, for example, migration/contraction, matrix metalloproteases (MMP) production, proliferation, and/or angiogenesis. There can be contraction (downsizing of the wound), epithelialization (creation of new epithelial cells) and/or deposition of connective tissue in order to heal the wound. Disclosed herein are compositions, methods and processes related to wound healing, i.e., the promotion and production of new skin cells/layers at a skin injury site, or the acceleration of wound healing.

In an adult, wounded healing can replace wounded tissues by scar tissues characterized by disorganized collagen structure, loss of hair follicles and/or irregular vascular structures when compared with normal skin. The composition disclosed herein can replace wounded tissues with normal skin tissues through the activities of the components, such as, the dermo-genic component, of the composition.

Acceleration of wound healing can be described by percentage (%) acceleration of wound healing and/or a Hazard ratio. In certain embodiments, the administration of the composition can accelerate wound healing equal to or greater than 10%, equal to or greater than 20%, equal to or greater than 30%, equal to or greater than 40%, equal to or greater than 50%, equal to or greater than 60%, equal to or greater than 70%, equal to or greater than 75%, equal to or greater than 80%, equal to or greater than 85%, equal to or greater than 90%, equal to or greater than 95%, equal to or greater than 100%, equal to or greater than 110% or more, when compared to a control without administering the composition. In some embodiments, the administration of the composition can accelerate wound healing between about10% and about 100%, between about 110% and about 200%, between about 200% and about 300%, and between about 300% and 400%, when compared to a control without administering the composition. In some embodiments, acceleration of wound healing can be described by a Hazard ratio, which is equal to or greater than 1.50, equal to or greater than 1.55, equal to or greater than 1.60, equal to or greater than 1.65, equal to or greater than 1.70, equal to or greater than 1.75, equal to or greater than 1.80, equal to or greater than 1.85, equal to or greater than 1.95, equal to or greater than 2.0, equal to or greater than 2.1, equal to or greater than 2.2, or equal to or greater than 2.3. In some embodiments, acceleration of wound healing is described by a Hazard ratio, which is between 1.50 and 2.3.

Disclosed herein are also compositions, methods and processes applicable to subjects who are undergoing or have undergone a treatment. In some cases, these treatments can delay or provide ineffective wound healing to the subjects. In some cases, these treatments can include, but are not limited to, medications, radiation, treatments that results in suppressed immune systems, etc. Optionally, a subject of the invention can have a secondary condition, wherein the secondary conditions can delay or provide ineffective wound healing. These secondary conditions, can include, but are not limited to, e.g., diabetes, peripheral vascular disease, infection, autoimmune or collagen vascular disorders, disease states that can result in suppressed immune systems, etc. The compositions disclosed herein can repair, replace, and/or reconstruction skin defects caused by injury, disease, wounds, or surgery.

### Composition

Disclosed herein are compositions for treating a skin condition. The composition can comprise: (a) an isolated and processed birthing tissue comprising an umbilical cord tissue, wherein said birthing tissue comprises from about 10% to about 50% by weight of said composition, and (b) at least one of: (i) a plurality of isolated stem cells, (ii) an isolated inductive component comprising a portion of a Wharton's jelly, (iii) a viscosity modifying component, and (iv) a cryopreservative.

The composition can support or promote skin wound healing through a dermo-conductive, dermo-inductive, dermo-genic and dermo-promoting mechanism to the local injury site. The composition can comprise (a) a dermo-conductive component, and (b) at least two of: (i) a dermo-genic component, (ii) a dermo-inductive component, and (iii) a dermo-promoting component.

A dermo-conductive component can serve as a scaffold for new skin tissue to attach, migrate, grow and/or divide. It can maintain space to allow for new skin cells/layers formation. Dermo-conductive mechanism or dermo-conductivity as used herein can refer to the ability of newly formed skin cells to move across a scaffold or framework and/or slowly replace the scaffold or framework with new skin tissues over time. The dermo-conductive component can serve as an adaptive template or substance on which skin cells/layers can grow. The dermo-conductive component can be an isolated scaffolding component from a subject, a mammal, or a human. The dermo-conductive component can be a birthing tissue. The dermo-conductive component can be an isolated and processed birthing tissue comprising an umbilical cord tissue. The dermo-conductive component can be umbilical cord tissues or derivatives thereof to provide a 3-dimensional framework upon which to spread and generate new skin tissues. A wound healing composition can be dermo-conductive. The dermo-conductive component can serves as a matrix to facilitate the infiltration of skin cells or other cells to the skin defect site, such as a wound bed. The dermo-conductive component can have surfaces, pores, channels or pipes. The dermo-conductive component can furnish a microenvironment that facilitates deposition or adhesion of dermo-genic cells.

A dermo-inductive component can involve the stimulation of progenitor cells and/or dermo-genic cells including stem cells to differentiate into an ensemble of skin cells to form the skin layers: the epidermis, the dermis and the hypodermi. In a dermo-inductive process, skin-forming cells can be recruited to the injury site and be induced to form new skin cells at the injury site. Dermo-inductive mechanism or dermo-inductivity as used herein can refer to the ability of a component to send a signal to attract, proliferate and differentiate early-lineage cells, for example, mesenchymal stem cells (MSCs) into skin-forming cells. Dermo-inductivity can recruit skin-forming cells to the skin defect site and induce the recruited skin-forming cells to a dermo-genic phenotype, i.e., a cell type for the three skin layers. The dermo-inductive component can be an isolated inductive component from a subject, a mammal, or a human. For example, the dermo-inductive component can be Wharton's Jelly or derivatives thereof to trigger the formation of new skin cells/layers and promote faster integration of such new skin cells/layers with existing skin cells/layers. The dermo-inductive component can comprise keratinocytes, fibroblasts or a combination thereof. The dermo-inductive component can recruit cells to the skin defect site, for example, a wound bed, and induce the skin-cell forming or healing ability of the recruited cells. Dermo-inductive chemicals generated by and/or released from the dermo-inductive component can promote and stimulate formation of skin cells.

A dermo-genic component can contribute to new skin growth along with the dermo-conductive and dermo-inductive mechanisms described above. Dermo-genic mechanism or dermo-genesis as used herein can refer to the development and formation of new cellular elements for the skin growth, thereby synthesizing new skin at the injury site. The dermo-genic component can be adapted to assist in wound healing. It can be non-immunogenic. The dermo-genic component can be a plurality of skin-forming cells. The dermo-genic component can be a plurality of isolated stem cells from a subject, a mammal, or a human. The dermo-genic component can be a number of skin-forming cells, such as stem cells, to form new skin and remodel the newly generated skin cells/layers into a viable skin. For example, the dermo-genic component can be mesenchymal stem cells (MSCs) or derivatives thereof, which are adapted to respond to their environment and differentiate into a variety of cell types as needed to form a new skin.

A dermo-promoting component can involve the enhancement of dermo-induction without the possession of dermo-inductive properties itself For example, a viscosity modifying component can increase the overall function of dermo-inductive component without displaying detectable dermo-inductivity itself in the absence of a dermo-inductive component. The dermo-promoting component can be adapted to facilitate in vivo use and formation of an effective configuration of other components of the wound healing composition disclosed herein. The dermo-promoting component can be a viscosity modifying component to provide a physical form adapted to perform the dermo-induction. The dermo-promoting component can exhibit a viscosity adapted to maintain the suitable physical form to be applied to the skin defect, such as a wound. For example, the dermo-promoting component can stabilize the dermo-inductive component at the skin defect, for example, a wound site, by preventing or slowing down the digestion of the dermo-inductive component by an enzyme, such as a proteinase.

### Viscosity Modifying Component

The composition can comprise a viscosity modifying component. In some cases, the viscosity modifying component can comprise a polyol, a sugar, a derivative of any of these, a salt of any of these, or any combination thereof. In some cases, the viscosity modifying component can comprise a polyol, a derivative thereof, a salt of any of these, or any combination thereof. In some cases, the polyol, the derivative thereof, or the salt of any of these can be a polyhydric alcohol, a derivative thereof, or a salt of any of these. In some cases, the polyhydric alcohol, said derivative thereof, or said salt of any of these can be a glycerol, an erythritol, a threitol, an arabitol, a xylitol, a ribitol, a mannitol, a sorbitol, a galactitol, a fucitol, an iditol, an inositol, a volemitol, an isomalt, a maltitol, a lactitol, a maltotriitol, a maltotetraitol, a polyglycitol, a C₃-C₃₀ polyhydric alcohol, a salt of any of these, a derivative of any of these, or any combination thereof. In some cases, the viscosity modifying component described herein can be alkylated, acetylated, phosphorylated, ubiquitinated, glycosylated, methylated, hydroxylated, sulfated, sulfonated, pegylated, propylene glycolated, contain an amino group, contain an imino group, or can be derived by any combination thereof.

In some cases, the viscosity modifying component can comprise a polymer. In some cases, the polymer can comprise a plurality of sugar monomers. In some cases, the polymer can comprise an alginic acid or a salt thereof. In some cases, the polymer can be selected from the group consisting of: carboxylic acid/carboxylate copolymers, acrylate homopolymers, acrylate copolymers, and any combination thereof. In some cases, the viscosity modifying component can comprise a carboxylic acid or a salt thereof. In some cases, the viscosity modifying component can comprise a gelatin or a derivative thereof. In some cases, the viscosity modifying component can comprise collagen. In some cases, the collagen can be at least partially hydrolyzed. In some cases, the polymer can be selected from the group consisting of: carboxylic acid/carboxylate copolymers, acrylate homopolymers, acrylate copolymers, and any combination thereof.

In some cases, the viscosity modifying component can be present in a proportion of from about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 11%, from about 1% to about 12%, about 1% to about 13%, about 1% to about 14%, about 1% to about 15%, about 1% to about 16%, about 1% to about 17%, about 1% to about 18%, about 1% to about 19%, about 1% to about 20% about 1% to about 22%, about 1% to about 23%, about 1% to about 24%, about 1% to about 25%, about 1% to about 26%, about 1% to about 27%, about 1% to about 28%, about 1% to about 29%, about 1% to about 30%, by weight of the composition described herein.

In some cases, the viscosity modifying component can be present in a proportion of from about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 0.10%, about 0.1% to about 0.11%, from about 0.1% to about 0.12%, about 0.1% to about 0.13%, about 0.1% to about 0.14%, about 0.1% to about 0.15%, about 0.1% to about 0.16%, about 0.1% to about 0.17%, about 0.1% to about 0.18%, about 0.1% to about 0.19%, about 0.1% to about 20% about 0.1% to about 22%, about 0.1% to about 23%, about 0.1% to about 24%, about 0.1% to about 25%, about 0.1% to about 26%, about 0.1% to about 27%, about 0.1% to about 28%, about 0.1% to about 29%, about 0.1% to about 30%, by weight of the composition described herein.

In some cases, the viscosity modifying component can be present in a proportion of from about 9% to about 17%, about 10% to about 17%, about 11% to about 17%, about 12% to about 17%, from about 9% to about 18%, about 10% to about 18%, about 11% to about 18%, about 12% to about 18%, about 13% to about 18%, from about 9% to about 19%, about 10% to about 19%, about 11% to about 19%, about 12% to about 19%, about 13% to about 19%, about 14% to about 19%, from about 9% to about 20%, about 10% to about 20%, about 11% to about 20%, about 12% to about 20%, about 13% to about 20%, about 14% to about 20%, or about 15% to about 20% by weight of the composition described herein.

In some cases, the viscosity modifying component can be present in a proportion of from about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 1% to about 11%, from about 1% to about 12%, about 1% to about 13%, about 1% to about 14%, about 1% to about 15%, about 1% to about 16%, about 1% to about 17%, about 1% to about 18%, about 1% to about 19%, about 1% to about 20% about 1% to about 22%, about 1% to about 23%, about 1% to about 24%, about 1% to about 25%, about 1% to about 26%, about 1% to about 27%, about 1% to about 28%, about 1% to about 29%, about 1% to about 30%, by volume of the composition described herein.

In some cases, the viscosity modifying component can be present in a proportion of from about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 0.10%, about 0.1% to about 0.11%, from about 0.1% to about 0.12%, about 0.1% to about 0.13%, about 0.1% to about 0.14%, about 0.1% to about 0.15%, about 0.1% to about 0.16%, about 0.1% to about 0.17%, about 0.1% to about 0.18%, about 0.1% to about 0.19%, about 0.1% to about 20% about 0.1% to about 22%, about 0.1% to about 23%, about 0.1% to about 24%, about 0.1% to about 25%, about 0.1% to about 26%, about 0.1% to about 27%, about 0.1% to about 28%, about 0.1% to about 29%, about 0.1% to about 30%, by volume of the composition described herein.

In some cases, the viscosity modifying component can be present in a proportion of from about 9% to about 17%, about 10% to about 17%, about 11% to about 17%, about 12% to about 17%, from about 9% to about 18%, about 10% to about 18%, about 11% to about 18%, about 12% to about 18%, about 13% to about 18%, from about 9% to about 19%, about 10% to about 19%, about 11% to about 19%, about 12% to about 19%, about 13% to about 19%, about 14% to about 19%, from about 9% to about 20%, about 10% to about 20%, about 11% to about 20%, about 12% to about 20%, about 13% to about 20%, about 14% to about 20%, or about 15% to about 20% by volume of the composition described herein.

The viscosity modifying component, when mixed with other components of the composition and a solvent, such as water, can modify the viscosity of the composition thus formed. The viscosity of the composition thus obtained is adapted to administer the composition to the subject at the skin defective site.

### Isolated Stem Cells

The composition can comprise a plurality of isolated stem cells. In some cases, the plural of isolated stem cells can be derived from umbilical cord blood and/or umbilical cord tissues. In some cases, the plurality of isolated stem cells can comprise mesenchymal stem cells (MSCs), hematopoietic stem cells (HSCs), or combination thereof. In some cases, the plurality of isolated stem cells can comprise mesenchymal stem cells (MSCs). MSCs can be multipotent stromal cells that can differentiate into a variety of cell types, such as osteoblasts (bone cells), chondrocytes (cartilage cells), myocytes (muscle cells) and adipocytes (fat cells). MSCs can be derived from bone marrow, umbilical cord tissue, adipose tissue, molar cells, amniotic fluid, or peripheral blood. For example, MSCs can be derived from umbilical cord tissue, such as Wharton's jelly and the umbilical cord blood. MSCs can also differentiate into a variety of skin cell types, such as, cells in epidermis (e.g., keratinocytes, melanocytes, Merkel cells, and Langerhans cells), cells in dermis (e.g., mast cells, vascular smooth muscle cells, specialized muscle cells, fibroblasts, and immune cells), and cells in hypodermis (e.g., adipocytes, nerve cells, macrophages, and fibroblasts). In some cases, MSCs can be extracted from umbilical cord tissues. In some cases, MSCs can be cryopreserved.

MSCs can be positive for cell surface markers CD105, CD166, CD90, and CD44. MSCs can be negative for hematopoietic antigens, such as CD45, CD34, and CD14.

In some cases, the plurality of isolated stem cells can comprise hematopoietic stem cells (HSCs). HSCs can give rise to all blood cells through the process of haematopoiesis. HSCs can be derived from mesoderm. HSCs can located in the red bone marrow, which is contained in the core of most bones.

In some cases, the plurality of isolated stem cells can be isolated from a birthing tissue. For example, the plurality of isolated stem cells can be isolated from a cord tissue, a placenta tissue, Wharton's jelly, or any combinations thereof. The isolated stem cells can be isolated from sources other than a birthing tissue.

In some cases, the plurality of isolated stem cells can comprise an autologous stem cell. In some cases, the plurality of isolated stem cells can comprise a heterologous stem cell. In some cases, at least a portion of the plurality of stem cells is viable. In some cases, the viable portion of the plurality of stem cells can be at least about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%,1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by weight relative to a total weight of a the plurality of stem cells.

In some cases, the plurality of isolated stem cells can be present in a proportion of from about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, or about 0.1% to about 10% by weight relative to a total weight of the composition.

In some cases, the plurality of isolated stem cells can be present in a proportion of from about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, or about 0.01% to about 10% by weight relative to a total weight of the composition.

In some cases, the plurality of isolated stem cells can be present in a proportion of from about 0.1% to about 1%, about 0.2% to about 1%, about 0.3% to about 1%, about 0.4% to about 1%, about 0.5% to about 1%, about 0.1% to about 2%, about 0.2% to about 2%, about 0.3% to about 2%, about 0.4% to about 2%, about 0.5% to about 2%, about 0.6% to about 2%, about 0.8% to about 2%, about 1% to about 2%, about 0.1% to about 3%, about 0.2% to about 3%, about 0.3% to about 3%, about 0.4% to about 3%, about 0.5% to about 3%, about 0.6% to about 3%, about 0.8% to about 3%, about 1% to about 3%, about 1.5% to about 3%, about 2% to about 3%, about 0.1% to about 4%, about 0.2% to about 4%, about 0.3% to about 4%, about 0.4% to about 4%, about 0.5% to about 4%, about 0.6% to about 4%, about 0.8% to about 4%, about 1% to about 4%, about 1.5% to about 4%, about 2% to about 4%, about 2.5% to about 4%, about 3% to about 4%, about 0.1% to about 5%, about 0.2% to about 5%, about 0.3% to about 5%, about 0.4% to about 5%, about 0.5% to about 5%, about 0.6% to about 5%, about 0.8% to about 5%, about 1% to about 5%, about 1.5% to about 5%, about 2% to about 5%, about 2.5% to about 5%, about 3% to about 5%, about 3.5% to about 5%, about 4% to about 5%, about 0.1% to about 6%, about 0.2% to about 6%, about 0.3% to about 6%, about 0.4% to about 6%, about 0.6% to about 6%, about 0.6% to about 6%, about 0.8% to about 6%, about 1% to about 6%, about 1.6% to about 6%, about 2% to about 6%, about 2.6% to about 6%, about 3% to about 6%, about 3.6% to about 6%, about 4% to about 6%, about 0.1% to about 7%, about 0.2% to about 7%, about 0.3% to about 7%, about 0.4% to about 7%, about 0.7% to about 7%, about 0.6% to about 7%, about 0.8% to about 7%, about 1% to about 7%, about 1.7% to about 7%, about 2% to about 7%, about 2.7% to about 7%, about 3% to about 7%, about 3.7% to about 7%, or about 4% to about 7%, by weight relative to a total weight of the composition.

In some cases, the viable portion of the plurality of stem cells can be at least about 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, 0.5%, 0.51%, 0.52%, 0.53%, 0.54%, 0.55%, 0.56%, 0.57%, 0.58%, 0.59%, 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, 0.9%, 0.91%, 0.92%, 0.93%, 0.94%, 0.95%, 0.96%, 0.97%, 0.98%, 0.99%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% by volume relative to a total volume of a the plurality of stem cells.

In some cases, the plurality of isolated stem cells can be present in a proportion of from about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, or about 0.1% to about 10% by volume relative to a total volume of the composition.

In some cases, the plurality of isolated stem cells can be present in a proportion of from about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, or about 0.01% to about 10% by volume relative to a total volume of the composition.

In some cases, the plurality of isolated stem cells can be present in a proportion of from about 0.1% to about 1%, about 0.2% to about 1%, about 0.3% to about 1%, about 0.4% to about 1%, about 0.5% to about 1%, about 0.1% to about 2%, about 0.2% to about 2%, about 0.3% to about 2%, about 0.4% to about 2%, about 0.5% to about 2%, about 0.6% to about 2%, about 0.8% to about 2%, about 1% to about 2%, about 0.1% to about 3%, about 0.2% to about 3%, about 0.3% to about 3%, about 0.4% to about 3%, about 0.5% to about 3%, about 0.6% to about 3%, about 0.8% to about 3%, about 1% to about 3%, about 1.5% to about 3%, about 2% to about 3%, about 0.1% to about 4%, about 0.2% to about 4%, about 0.3% to about 4%, about 0.4% to about 4%, about 0.5% to about 4%, about 0.6% to about 4%, about 0.8% to about 4%, about 1% to about 4%, about 1.5% to about 4%, about 2% to about 4%, about 2.5% to about 4%, about 3% to about 4%, about 0.1% to about 5%, about 0.2% to about 5%, about 0.3% to about 5%, about 0.4% to about 5%, about 0.5% to about 5%, about 0.6% to about 5%, about 0.8% to about 5%, about 1% to about 5%, about 1.5% to about 5%, about 2% to about 5%, about 2.5% to about 5%, about 3% to about 5%, about 3.5% to about 5%, about 4% to about 5%, about 0.1% to about 6%, about 0.2% to about 6%, about 0.3% to about 6%, about 0.4% to about 6%, about 0.6% to about 6%, about 0.6% to about 6%, about 0.8% to about 6%, about 1% to about 6%, about 1.6% to about 6%, about 2% to about 6%, about 2.6% to about 6%, about 3% to about 6%, about 3.6% to about 6%, about 4% to about 6%, about 0.1% to about 7%, about 0.2% to about 7%, about 0.3% to about 7%, about 0.4% to about 7%, about 0.7% to about 7%, about 0.6% to about 7%, about 0.8% to about 7%, about 1% to about 7%, about 1.7% to about 7%, about 2% to about 7%, about 2.7% to about 7%, about 3% to about 7%, about 3.7% to about 7%, or about 4% to about 7%, by volume relative to a total volume of the composition.

### Isolated Inductive Component

The composition can comprise an isolated inductive component. In some cases, the isolated inductive component can comprise a portion of a Wharton's jelly. In some cases, Wharton's jelly can be a gelatinous substance derived from an umbilical cord. In some cases, Wharton's jelly can comprise mucopolysaccharides (e.g., hyaluronic acid, chondroitin sulfate), proteoglycans, fibroblasts and macrophages. Wharton's jelly can comprise extracellular matrix components that are composed primarily of collagen, hyaluronic acid, and various sulphated proteoglycans. The biosynthesis of such extracellular matrix components can be enhanced by peptide growth factors, including IGF, FGF and TGF-beta. These growth factors can accumulate within Wharton's jelly and affects other cells, e.g. MSCs.

In some cases, Wharton's jelly can comprise myofibroblast-like stromal cells (umbilical cord stromal cells). In some cases, Wharton's jelly can express several stem cell genes, including telomerase. In some cases, Wharton's jelly can supply adult stem cells. In some cases, Wharton's jelly can facilitate skin cell migration, accelerate the proliferation phase of wound healing, enhance differentiation of skin cells. In some cases, Wharton's jelly can be extracted from umbilical cord tissues. In some cases, Wharton's jelly can be cryopreserved after the extraction.

In some cases, the isolated inductive component can comprise a mucous tissue. In some cases, the isolated inductive component can comprise a mucopolysaccharide or a salt thereof. In some cases, the isolated inductive component can comprise hyaluronic acid or a salt thereof. In some cases, the isolated inductive component can comprise chondroitin or a salt thereof. In some cases, the isolated inductive component can comprise a salt of chondroitin. In some cases, the isolated inductive component can comprise N-acetylgalactoasmine or a salt thereof. In some cases, the isolated inductive component can comprise glucuronic acid or a salt thereof. In some cases, the isolated inductive component can comprise water. In some cases, the isolated inductive component can comprise a plurality of isolated fibroblasts. In some cases, at least a portion of the plurality of isolated fibroblasts can be viable. In some cases, the isolated inductive component can comprise a plurality of isolated macrophages. In some cases, at least a portion of the plurality of isolated macrophages is viable.

In some cases, the isolated inductive component can be present in a portion of from about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 11%, about 0.1% to about 12%, about 0.1% to about 13%, about 0.1% to about 14%, about 0.1% to about 15%, about 0.1% to about 16%, about 0.1% to about 17%, about 0.1% to about 18%, about 0.1% to about 19%, about 0.1% to about 20%, about 0.1% to about 21%, about 0.1% to about 22%, about 0.1% to about 23%, about 0.1% to about 24%, about 0.1% to about 25%, about 0.1% to about 26%, about 0.1% to about 27%, about 0.1% to about 28%, about 0.1% to about 29%, about 0.1% to about 30%, about 0.1% to about 31%, about 0.1% to about 32%, about 0.1% to about 33%, about 0.1% to about 34%, about 0.1% to about 35%, about 0.1% to about 36%, about 0.1% to about 37%, about 0.1% to about 38%, about 0.1% to about 39%, about 0.1% to about 40%, about 0.1% to about 41%, about 0.1% to about 42%, about 0.1% to about 43%, about 0.1% to about 44%, about 0.1% to about 45%, about 0.1% to about 46%, about 0.1% to about 47%, about 0.1% to about 48%, about 0.1% to about 49%, about 0.1% to about 50%, about 0.1% to about 51%, about 0.1% to about 52%, about 0.1% to about 53%, about 0.1% to about 54%, about 0.1% to about 55%, about 0.1% to about 56%, about 0.1% to about 57%, about 0.1% to about 58%, about 0.1% to about 59%, about 0.1% to about 60%, about 0.1% to about 61%, about 0.1% to about 62%, about 0.1% to about 63%, about 0.1% to about 64%, about 0.1% to about 65%, about 0.1% to about 66%, about 0.1% to about 67%, about 0.1% to about 68%, about 0.1% to about 69%, or about 0.1% to about 70% by weight of the composition.

In some cases, the isolated inductive component can be present in a portion of from about 1% to about 50%, about 2% to about 50%, about 3% to about 50%, about 4% to about 50%, about 5% to about 50%, about 6% to about 50%, about 7% to about 50%, about 8% to about 50%, about 9% to about 50%, about 10% to about 50%, about 11% to about 50%, about 12% to about 50%, about 13% to about 50%, about 14% to about 50%, about 15% to about 50%, about 16% to about 50%, about 17% to about 50%, about 18% to about 50%, about 19% to about 50%, about 20% to about 50%, 21% to about 50%, about 22% to about 50%, about 23% to about 50%, about 24% to about 50%, about 25% to about 50%, about 26% to about 50%, about 27% to about 50%, about 28% to about 50%, about 29% to about 50%, or 30% to about 50% by weight of the composition.

In some cases, the isolated inductive component can be present in a portion of from about 1% to about 60%, about 2% to about 60%, about 3% to about 60%, about 4% to about 60%, about 5% to about 60%, about 6% to about 60%, about 7% to about 60%, about 8% to about 60%, about 9% to about 60%, about 10% to about 60%, about 11% to about 60%, about 12% to about 60%, about 13% to about 60%, about 14% to about 60%, about 15% to about 60%, about 16% to about 60%, about 17% to about 60%, about 18% to about 60%, about 19% to about 60%, about 20% to about 60%, 21% to about 60%, about 22% to about 60%, about 23% to about 60%, about 24% to about 60%, about 25% to about 60%, about 26% to about 60%, about 27% to about 60%, about 28% to about 60%, about 29% to about 60%, or 30% to about 60% by weight of the composition.

In some cases, the isolated inductive component can be present in a portion of from about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 11%, about 0.1% to about 12%, about 0.1% to about 13%, about 0.1% to about 14%, about 0.1% to about 15%, about 0.1% to about 16%, about 0.1% to about 17%, about 0.1% to about 18%, about 0.1% to about 19%, about 0.1% to about 20%, about 0.1% to about 21%, about 0.1% to about 22%, about 0.1% to about 23%, about 0.1% to about 24%, about 0.1% to about 25%, about 0.1% to about 26%, about 0.1% to about 27%, about 0.1% to about 28%, about 0.1% to about 29%, about 0.1% to about 30%, about 0.1% to about 31%, about 0.1% to about 32%, about 0.1% to about 33%, about 0.1% to about 34%, about 0.1% to about 35%, about 0.1% to about 36%, about 0.1% to about 37%, about 0.1% to about 38%, about 0.1% to about 39%, about 0.1% to about 40%, about 0.1% to about 41%, about 0.1% to about 42%, about 0.1% to about 43%, about 0.1% to about 44%, about 0.1% to about 45%, about 0.1% to about 46%, about 0.1% to about 47%, about 0.1% to about 48%, about 0.1% to about 49%, about 0.1% to about 50%, 0.1% to about 51%, about 0.1% to about 52%, about 0.1% to about 53%, about 0.1% to about 54%, about 0.1% to about 55%, about 0.1% to about 56%, about 0.1% to about 57%, about 0.1% to about 58%, about 0.1% to about 59%, about 0.1% to about 60%, by weight of the composition.

In some cases, the isolated inductive component can be present in a portion of from about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 11%, about 0.1% to about 12%, about 0.1% to about 13%, about 0.1% to about 14%, about 0.1% to about 15%, about 0.1% to about 16%, about 0.1% to about 17%, about 0.1% to about 18%, about 0.1% to about 19%, about 0.1% to about 20%, about 0.1% to about 21%, about 0.1% to about 22%, about 0.1% to about 23%, about 0.1% to about 24%, about 0.1% to about 25%, about 0.1% to about 26%, about 0.1% to about 27%, about 0.1% to about 28%, about 0.1% to about 29%, about 0.1% to about 30%, about 0.1% to about 31%, about 0.1% to about 32%, about 0.1% to about 33%, about 0.1% to about 34%, about 0.1% to about 35%, about 0.1% to about 36%, about 0.1% to about 37%, about 0.1% to about 38%, about 0.1% to about 39%, about 0.1% to about 40%, about 0.1% to about 41%, about 0.1% to about 42%, about 0.1% to about 43%, about 0.1% to about 44%, about 0.1% to about 45%, about 0.1% to about 46%, about 0.1% to about 47%, about 0.1% to about 48%, about 0.1% to about 49%, about 0.1% to about 50%, about 0.1% to about 51%, about 0.1% to about 52%, about 0.1% to about 53%, about 0.1% to about 54%, about 0.1% to about 55%, about 0.1% to about 56%, about 0.1% to about 57%, about 0.1% to about 58%, about 0.1% to about 59%, about 0.1% to about 60%, about 0.1% to about 61%, about 0.1% to about 62%, about 0.1% to about 63%, about 0.1% to about 64%, about 0.1% to about 65%, about 0.1% to about 66%, about 0.1% to about 67%, about 0.1% to about 68%, about 0.1% to about 69%, or about 0.1% to about 70% by volume of the composition.

In some cases, the isolated inductive component can be present in a portion of from about 1% to about 50%, about 2% to about 50%, about 3% to about 50%, about 4% to about 50%, about 5% to about 50%, about 6% to about 50%, about 7% to about 50%, about 8% to about 50%, about 9% to about 50%, about 10% to about 50%, about 11% to about 50%, about 12% to about 50%, about 13% to about 50%, about 14% to about 50%, about 15% to about 50%, about 16% to about 50%, about 17% to about 50%, about 18% to about 50%, about 19% to about 50%, about 20% to about 50%, 21% to about 50%, about 22% to about 50%, about 23% to about 50%, about 24% to about 50%, about 25% to about 50%, about 26% to about 50%, about 27% to about 50%, about 28% to about 50%, about 29% to about 50%, or 30% to about 50% by volume of the composition.

In some cases, the isolated inductive component can be present in a portion of from about 1% to about 60%, about 2% to about 60%, about 3% to about 60%, about 4% to about 60%, about 5% to about 60%, about 6% to about 60%, about 7% to about 60%, about 8% to about 60%, about 9% to about 60%, about 10% to about 60%, about 11% to about 60%, about 12% to about 60%, about 13% to about 60%, about 14% to about 60%, about 15% to about 60%, about 16% to about 60%, about 17% to about 60%, about 18% to about 60%, about 19% to about 60%, about 20% to about 60%, 21% to about 60%, about 22% to about 60%, about 23% to about 60%, about 24% to about 60%, about 25% to about 60%, about 26% to about 60%, about 27% to about 60%, about 28% to about 60%, about 29% to about 60%, or 30% to about 60% by volume of the composition.

In some cases, the isolated inductive component can be present in a portion of from about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 11%, about 0.1% to about 12%, about 0.1% to about 13%, about 0.1% to about 14%, about 0.1% to about 15%, about 0.1% to about 16%, about 0.1% to about 17%, about 0.1% to about 18%, about 0.1% to about 19%, about 0.1% to about 20%, about 0.1% to about 21%, about 0.1% to about 22%, about 0.1% to about 23%, about 0.1% to about 24%, about 0.1% to about 25%, about 0.1% to about 26%, about 0.1% to about 27%, about 0.1% to about 28%, about 0.1% to about 29%, about 0.1% to about 30%, about 0.1% to about 31%, about 0.1% to about 32%, about 0.1% to about 33%, about 0.1% to about 34%, about 0.1% to about 35%, about 0.1% to about 36%, about 0.1% to about 37%, about 0.1% to about 38%, about 0.1% to about 39%, about 0.1% to about 40%, about 0.1% to about 41%, about 0.1% to about 42%, about 0.1% to about 43%, about 0.1% to about 44%, about 0.1% to about 45%, about 0.1% to about 46%, about 0.1% to about 47%, about 0.1% to about 48%, about 0.1% to about 49%, about 0.1% to about 50%, 0.1% to about 51%, about 0.1% to about 52%, about 0.1% to about 53%, about 0.1% to about 54%, about 0.1% to about 55%, about 0.1% to about 56%, about 0.1% to about 57%, about 0.1% to about 58%, about 0.1% to about 59%, about 0.1% to about 60%, by volume of the composition.

### Isolated Scaffolding Component

The composition can comprise an isolated scaffolding component. In some cases, the isolated scaffolding component can comprise a birthing tissue. In some cases, the birthing tissue is isolated and processed. In some cases, the birthing tissue can comprise an umbilical cord tissue, a placental tissue, or a combination thereof. In some cases, the isolated and processed birthing tissue can comprise from about 10% to about 50% by weight of the composition. In some cases, the isolated scaffolding component can comprise a conductive property. In some cases, the isolated and processed birthing tissue can comprise a conductive property. In some cases, the isolated and processed birthing tissue can comprise minced tissue. In some cases, the isolated and processed birthing tissue can comprise micronized tissue. In some cases, the isolated and processed birthing tissue can comprise lyophilized tissue. In some cases, the isolated and processed birthing tissue can comprise a plurality of pieces of tissue.

The isolated scaffolding component can be used to direct regenerative events to form biologically and mechanically responsive tissues. In some cases, the isolated scaffolding component can be an isolated and processed birthing tissue. A birthing tissue as used herein can refer to cord tissues including Wharton's jelly, placenta, amnion, exocoelomic space, chorion, decidual capsularis, etc., or any combinations thereof.

When attached to both a pregnant woman and her fetus, an umbilical cord can provide an uninterrupted flow of blood from the mother's placenta to the fetus during the fetus' development in the womb. Umbilical cord tissue can comprise many different types of cells, including fat (e.g., Wharton's jelly), white cells, and stem cells such as MSCs, epithelial stem cells, and endothelial stem cells. MSCs, as disclosed elsewhere, can help regenerating damaged body tissues, including skin tissues. Other than stem cells, umbilical cord tissues can provide three-dimensional scaffolding for newly generated cells/tissues to grow upon. Cord tissues or umbilical cord tissues can be the insulating material surrounding the blood vessels of the umbilical cord. After delivery, the umbilical cord can be clamped and cut, processed or preserved.

The maternal and fetal blood can be separated by a placental membrane. The maternal-fetal exchange of nutrients and gases, etc., can be provided by villi within the placenta. The placenta can comprise two parts. One can be the basal plate which is of maternal origin and has tissues and cells that are immunologically or genetically identical to the mother. The other can be a chorionic plate which is of embryonic origin, the fetal portion. An umbilical cord can connect the fetus to the placenta of the mother, and carry two umbilical arteries and a single umbilical vein.

Placenta can be a source of cells, including stem cells such as MSCs, blood-forming stem cells and endothelial progenitor cells, etc. Similar to umbilical cord blood and umbilical cord tissue, placenta can be kept under low temperatures, or to be cryopreserved. As disclose elsewhere, MSCs can differentiate into different cell types including cartilage cells, bone, skin, fat, muscle, and heart cells. Blood-forming stem cells can form blood cells. Endothelial progenitor cells can help forming new blood vessels or repairing blood cells.

In some cases, the birthing tissue can be processed. In some cases, the birthing tissue can be a processed birthing tissue. The processed birthing tissue can be mechanically processed, chemically processed, freeze/thaw processed, or any combination thereof. In some cases, the processed birthing tissue can be at least two of: mechanically processed, chemically processed, and freeze/thaw processed. In some cases, the birthing tissue can be extracted to remove certain cells/tissues/chemicals. In some cases, the birthing tissue can be minced or micronized. In some cases, the birthing tissue can be cryopreserved. In some cases, the frozen birthing tissue can be thawed at temperatures higher than the freezing point of the solvent(s) used to freeze the birthing tissue. In some cases, the birthing tissue can be treated with a chemical reagent for immunological purposes or other purposes.

In some cases, the processed birthing tissue can further comprise a plurality of cells. In some cases, the processed birthing tissue can further comprise a plurality of viable cells. In some cases, the processed birthing tissue can comprise a plurality of stem cells. In some cases, the processed birthing tissue can comprise a portion of Wharton's jelly.

Decellularization can be the process used to isolate the extracellular matrix (ECM) of a tissue from its inhabiting cells, leaving an ECM scaffold of the original tissue, which can be used in artificial organ and tissue regeneration. The decellularization process can create a natural biomaterial to act as a scaffold for cell growth, differentiation and/or tissue development. By recellularizing an ECM scaffold with a patient's own cells, adverse immune responses can be eliminated. In some cases, the birthing tissue may not be decellularized. In some cases, the birthing tissue may be decellularized. In some cases, the birthing tissue can be physically, chemically, and/or enzymatically treated. In some cases, the physical treatment can lyse, kill, and/or remove cells from the matrix of a tissue through the use of temperature, force and pressure, and/or electrical disruption. In some cases, the chemical treatment can lyse, kill, and/or remove cells using an acid, an alkaline, an ionic detergent, a non-ionic detergent, a zwitterionic detergent, or any combinations thereof. In some cases, the enzymatic treatment can lyse, kill, and/or remove cells using a lipase, thermolysin, a galactosidase, a nuclease, trypsin, or any combinations thereof.

In some cases, the umbilical cord tissue can be processed. In some cases, the process can comprise extraction of certain component of the umbilical cord tissues. In some cases, the process can comprise extraction of MSCs. In some cases, the process can comprise extraction of Wharton's Jelly. In some cases, the process can comprise mincing or micronizing umbilical cord tissues after the extractions. In some cases, the umbilical cord tissues can be cryopreserved after the extraction and mincing/micronizing processes. In some cases, minced/micronized umbilical cord tissues can be easier to handle in terms of preservation and the eventual formation of the composition to treat skin defects.

In some cases, the isolated and processed birthing tissue can be present in a portion of from about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 20% to about 21%, about 20% to about 22%, about 20% to about 23%, about 20% to about 24%, about 20% to about 25%, about 20% to about 26%, about 20% to about 27%, about 20% to about 28%, about 20% to about 29%, about 20% to about 30%, about 20% to about 31%, about 20% to about 32%, about 20% to about 33%, about 20% to about 34%, about 20% to about 35%, about 20% to about 36%, about 20% to about 37%, about 20% to about 38%, about 20% to about 39%, about 20% to about 40%, about 20% to about 41%, about 20% to about 42%, about 20% to about 43%, about 20% to about 44%, about 20% to about 45%, about 20% to about 46%, about 20% to about 47%, about 20% to about 48%, about 20% to about 49%, about 20% to about 50%, 20% to about 51%, about 20% to about 52%, about 20% to about 53%, about 20% to about 54%, about 20% to about 55%, about 20% to about 56%, about 20% to about 57%, about 20% to about 58%, about 20% to about 59%, about 20% to about 60%, by weight of the composition.

In some cases, the isolated and processed birthing tissue can be present in a portion of from about 1% to about 50%, about 2% to about 50%, 3% to about 50%, 4% to about 50%, 5% to about 50%, 6% to about 50%, 7% to about 50%, 8% to about 50%, 9% to about 50%, 10% to about 50%, 11% to about 50%, 12% to about 50%, 13% to about 50%, 14% to about 50%, 15% to about 50%, 16% to about 50%, 17% to about 50%, 18% to about 50%, 19% to about 50%, about 20% to about 50%, 21% to about 50%, about 22% to about 50%, about 23% to about 50%, about 24% to about 50%, about 25% to about 50%, about 26% to about 50%, about 27% to about 50%, about 28% to about 50%, about 29% to about 50%, 30% to about 50%, 30% to about 51%, about 30% to about 52%, about 30% to about 53%, about 30% to about 54%, about 30% to about 55%, about 30% to about 56%, about 30% to about 57%, about 30% to about 58%, about 30% to about 59%, about 30% to about 60%, by weight of the composition.

In some cases, the isolated and processed birthing tissue can be present in a portion of from about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 20% to about 21%, about 20% to about 22%, about 20% to about 23%, about 20% to about 24%, about 20% to about 25%, about 20% to about 26%, about 20% to about 27%, about 20% to about 28%, about 20% to about 29%, about 20% to about 30%, about 20% to about 31%, about 20% to about 32%, about 20% to about 33%, about 20% to about 34%, about 20% to about 35%, about 20% to about 36%, about 20% to about 37%, about 20% to about 38%, about 20% to about 39%, about 20% to about 40%, about 20% to about 41%, about 20% to about 42%, about 20% to about 43%, about 20% to about 44%, about 20% to about 45%, about 20% to about 46%, about 20% to about 47%, about 20% to about 48%, about 20% to about 49%, about 20% to about 50%, 20% to about 51%, about 20% to about 52%, about 20% to about 53%, about 20% to about 54%, about 20% to about 55%, about 20% to about 56%, about 20% to about 57%, about 20% to about 58%, about 20% to about 59%, about 20% to about 60%, by volume of the composition.

In some cases, the isolated and processed birthing tissue can be present in a portion of from about 1% to about 50%, about 2% to about 50%, 3% to about 50%, 4% to about 50%, 5% to about 50%, 6% to about 50%, 7% to about 50%, 8% to about 50%, 9% to about 50%, 10% to about 50%, 11% to about 50%, 12% to about 50%, 13% to about 50%, 14% to about 50%, 15% to about 50%, 16% to about 50%, 17% to about 50%, 18% to about 50%, 19% to about 50%, about 20% to about 50%, 21% to about 50%, about 22% to about 50%, about 23% to about 50%, about 24% to about 50%, about 25% to about 50%, about 26% to about 50%, about 27% to about 50%, about 28% to about 50%, about 29% to about 50%, 30% to about 50%, 30% to about 51%, about 30% to about 52%, about 30% to about 53%, about 30% to about 54%, about 30% to about 55%, about 30% to about 56%, about 30% to about 57%, about 30% to about 58%, about 30% to about 59%, about 30% to about 60%, by volume of the composition.

### Combination of Components

In some embodiments, the composition can comprise a combination of components. In some cases, the composition can comprise at least two of: a viscosity agent, stem cells, Wharton's jelly, and a birthing tissue. In some cases, the composition can comprise a viscosity agent and stem cells. In some cases, the composition can comprise a viscosity agent and Wharton's jelly. In some cases, the composition can comprise a viscosity agent and a birthing tissue. In some cases, the composition can comprise a stem cells and Wharton's jelly. In some cases, the composition can comprise a stem cells and a birthing tissue. In some cases, the combination can comprise Wharton's jelly and a birthing tissue.

In some cases, the composition can comprise at least three of: a viscosity agent, stem cells, Wharton's jelly, and a birthing tissue. In some cases, the composition can comprise a viscosity agent, stem cells, and Wharton's jelly. In some cases, the composition can comprise a viscosity agent, stem cells, and a birthing tissue. In some cases, the composition can comprise a viscosity agent, Wharton's jelly, and a birthing tissue. In some cases, the composition can comprise stem cells, Wharton's jelly, and a birthing tissue.

In some cases, the composition can comprise (a) a viscosity agent, and (b) at least one of: stem cells, Wharton's jelly, and a birthing tissue. In some cases, the composition can comprise (a) stem cells, and (b) at least one of: a viscosity agent, Wharton's jelly, and a birthing tissue. In some cases, the composition can comprise (a) Wharton's jelly, and (b) at least one of: a viscosity agent, stem cells and a birthing tissue. In some cases, the composition can comprise (a) a birthing tissue, and (b) at least one of: a viscosity agent, stem cells, and Wharton's jelly.

In some cases, the composition can comprise (a) a viscosity agent, and (b) at least two of: stem cells, Wharton's jelly, and a birthing tissue. In some cases, the composition can comprise (a) stem cells, and (b) at least two of: a viscosity agent, Wharton's jelly, and a birthing tissue. In some cases, the composition can comprise (a) Wharton's jelly, and (b) at least one of: a viscosity agent, stem cells and a birthing tissue. In some cases, the composition can comprise (a) a birthing tissue, and (b) at least one of: a viscosity agent, stem cells, and Wharton's jelly.

In some cases, the composition can comprise a viscosity agent, stem cells, Wharton's jelly, and a birthing tissue.

### Other Components

The composition can further comprise a plurality of epithelial cells. The composition can further comprise a plurality of endothelial cells. The composition can further comprise a plurality of perivascular cells. The composition can further comprise a peptide. The composition can further comprise a protein. The composition can further comprise an amino acid. The composition can further comprise water.

The composition can further comprise at least one growth factor. In some cases, the at least one growth factor can comprise insulin-like growth factor-1, insulin-like growth factor binding protein-3, vascular endothelial growth factor (VEGF), hepatocyte growth factor (HGF), placenta growth factor (PLGF), or any combination thereof. The at least one growth factors can enhance viability, enhance stability of product, differentiation of cells, preservation of sternness, reduce anti-inflammatory, or any combinations thereof. The at least one growth factor can be added to the composition. The at least one growth factor can be added to a subcomponent of the composition. The at least one growth factor can be added to a viscosity modifying component, a plurality of isolated stem cells, an isolated inductive component, an isolated scaffolding component, or any combinations thereof. For example, the at least one growth factor can be added to a birthing tissue. The at least one growth factor can be added prior to forming the composition. The at least one growth factor can be added after forming the composition.

The composition can further comprise at least one of: chemokine ligand 2, macrophage inflammatory protein-1 (MIP-1) alpha, MIP-1 beta, MIP-2, beta-chemokine ligand-5, beta-chemokine ligand-20, alpha-chemokine ligand-14, lipopolysaccharide-induced alpha-chemokine, Granulocyte-macrophage colony-stimulating factor, interleukin IL-1 beta, phorbol myristate acetate, epidermal growth factor, fibroblast growth factor, vascular endothelial growth factor, connective tissue growth factor, platelet-derived growth factor, insulin-like growth factor, nerve growth factor, hepatocyte growth factor, colony-stimulating factor, stern cell factor, keratinocyte growth factor, granulocyte colony-stimulating factor, granulocyte macrophage colony-stimulating factor, glial derived neurotrophic factor, ciliary neurotrophic factor, endothelial-monocyte activating polypeptide, epithelial neutrophil activating peptide, erythropoietin, bone morphogenetic protein, brain-derived neurotrophic factor, transforming growth factor beta, tumor necrosis factor, or any combination thereof.

The composition can further comprise at least one hormone. In some cases, the at least one hormone can be prolactin or leptin.

The composition can further comprise a cellular cryopreservative. In some cases, the cellular cryopreservative can comprise a sucrose, a trehalose, a starch, a derivative of any of these, or any combination thereof.

In some cases, the compositions disclosed herein can combine with one or more of, for example, good wound care therapy (e.g., GWC), other wound therapies (for example, growth factors, nerve growth factor (NGF), positive angiogenesis factors or agents or activators, anabolic steroids, bioengineered tissue replacements (e.g., APLIGRAPH®, DERMAGRAF™, etc.), hyperbaric oxygen, or vacuum therapy, etc.) for enhancing the activity and/or efficiency of the compositions disclosed herein, in accelerating and/or improving wound healing.

In some cases, there can be six major growth factor families (EGF, FGF, IGF, PDGF, TGF, and VEGF) associated with wound healing. Examples of such growth factors can include, but are not limited to, platelet derived growth factor (PDGF-A, PDGF-B, PDGF-C, and PDGF-D), insulin-like growth factor I and II (IGF-I and IGF-II), acidic and basic fibroblast growth factor (aFGF and bFGF), alpha and beta transforming growth factor (TGF-α and TGF-β (for example, TGF-beta 1, TGF beta 2, TGF beta 3)), epidermal growth factor (EGF), and others. These growth factors can stimulate mitosis of one or more of the cells involved in wound healing and can be combined.

Other positive angiogenesis agents co-administered with the compositions disclosed herein can include, but are not limited to, e.g., HGF, TNF-α, angiogenin, IL-8, etc. Still further examples of additional agents can include Platelet-derived growth factor (PDGF) (e.g., Becaplermin (rhPDGF-BB) such as REGRANEX®, adenosine-A2A receptor agonists; keratinocyte growth factor (KGF-2, repifermin; lactoferrin (LF); thymosine beta-4 (Tβ4); thrombin-derived activating receptor peptide (TP508; CHRYSALIN®; adenoviral vector encoding platelet-derived growth factor (PDGF-B); autologous bone marrow stem cells (BMSC); and, engineered living tissue grafts (e.g., Apligraf, etc.). Antibiotic and antiseptic ulcer agents can also be combined. Immunosuppressive treatment (e.g., corticosteroids, radiation therapy, chemotherapy), other cancer treatment, or diabetic treatment can be combined with the compositions disclosed herein.

Additional agents can be co-administered with the composition disclosed herein or administered separately.

### Formulation

Disclosed herein are formulations for treating a skin condition. The relative amounts of viscosity modifying component, and at least one of: a plurality of isolated stem cells, an isolated inductive component, and an isolated scaffolding component; can be selected such that the composition can exhibit physical properties (e.g., deformability, pliability, viscosity, consistency, stability, etc.) that can be desired for the particular application. For example, by varying the relative amounts of the components, the final compositions can be formulated to be settable, moldable, injectable, sprayable, etc.

In some embodiments, the composition can be formulated such that the final composition can include about 1% to about 20% of a viscosity modifying component, about 0.1% to about 5% of a plurality of isolated stem cells, about 0.1% to about 50% of an isolated inductive component, and about 0.1% to about 50% isolated scaffolding component. In some embodiments, the composition can be formulated such that the final composition can include about 15% of a viscosity modifying component, about to 1% of a plurality of isolated stem cells, about 42% of an isolated inductive component, and about 42% isolated scaffolding component.

The composition can be in unit dose form. The composition can be formulated as a liquid, an aerosol, an aerosolized liquid, a foam, a cream, a gel, an ointment, a putty, a semi-solid, a glue, a coating, an adhesive, or any combination thereof.

The composition can comprise at 70° F. a viscosity from about 0.5 centipoise (cp) to about 2 cp. The composition can comprise at 70° F. a viscosity from about 100 cp to about 900 cp. The composition can comprise at 70° F. a viscosity from about cp to about 5,000 cp at 70° F. The composition can comprise at 70° F. a viscosity from about 50,000 cp to about 80,000 cp. The composition can comprise at 70° F. a viscosity from about 90,000 cp to about 150,000 cp. The composition can comprise at 70° F. a viscosity from about 200,000 to about 300,000 cp. The viscosity of the composition can be selected and controlled such that the composition can be formulated to be settable, moldable, injectable, sprayable, etc.

The composition can maintain at least about 90% of a wound healing activity after storage in a container at an average temperature from about 25 degrees Celsius to about 200 degrees Celsius. In some cases, the composition can maintain at least about 1%, about 2%, 3%, 4%, 5%, 6%, 7%, 8%, 9%, 10%, 11%, 12%, 13%, 14%, 15%, 16%, 17%, 18%, 19%, about 20%, about 21%, about 22%, about 23%, about 24%, about 25%, about 26%, about 27%, about 28%, about 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 8S%, 89%, 90% of a wound healing activity after storage in a container at an average temperature from about 25 degrees Celsius to about 200 degrees Celsius. The container is a sealed container.

### Cryopreservatives

The formulations can further comprise one or more cryopreservatives. Cryopreservation can preserve cells or whole tissues by cooling them to low sub-zero temperatures (e.g., lower than −90° C.). At such low temperatures, biological activities, including those biochemical reactions leading to cell death, can be effectively stopped or slowed. Methods can be used to preserve cells obtained from mammals, including humans. For example, cryopreservation of cells in a medium containing from about 20% to about 90% by weight of fetal bovine serum (FBS) and from about 10% to about 20% by weight of dimethyl sulfoxide (DMSO) as a cryopreservative can yield viable cells in cells recovery stage upon thawing. In some cases, controlled slow rate of freezing can minimize the formation of intracellular ice-crystals, which can contribute to frozen cell damage during the cryopreservation procedure. Rapid thawing of frozen cells at 37° C. can also improve viable cell recoveries.

To prepare the isolated cells for cryopreservation, the cells can be suspended in a culture medium comprising a cryoproservative before being dispensed into the cryopreservation receptacle. The cryopreservative can minimize the deleterious effects of cryopreservation, such as formation of intracellular ice. Suitable cryopreservatives can comprise fetal bovine serum (FBS), dimethyl sulfoxide (DMSO), polyethylene glycol, amino acids, polysaccharides (e.g., dextran, glucan, arabinogalactans, etc.), isopropyl alcohol, propanediol, glycerol, propylene glycol, sucrose, sodium glutamate, sorbitol, polyol (e.g. mannitol), trehalose, or a combination thereof. Other suitable cryopreservatives include CRYYOSTOR™ cryopreservation media, such as CS5 (5% DMSO), CS10 (10% DMSO), and CS2/DLITE® (2% DMSO), available from BIOLIFE SOLUTIONS®. The resulting suspension of cells for cryopreservation can have a viable cell concentration of from about 1 million cells/mL to about 20 million cells/mL, about 2 million cells/mL to about 19 million cells/mL, about 3 million cells/mL to about 18 million cells/mL, about 4 million cells/mL to about 17 million cells/mL, about 5 million cells/mL to about 16 million cells/mL, about 6 million cells/mL to about 15 million cells/mL, about 7 million cells/mL to about 15 million cells/mL, about 8 million cells/mL to about 15 million cells/mL, about 9 million cells/mL to about 15 million cells/mL, from about 10 million cells/mL to about 15 million cells/mL, about 11 million cells/mL to about 15 million cells/mL, about 11 million cells/mL to about 14 million cells/mL, or about 11 million cells/mL to about 13 million cells/mL. For subcellular fractions the viable concentration for cryopreservation can range from about 1 mg/mL to about 200 mg/mL, about 2 mg/mL to about 190 mg/mL, about 3 mg/mL to about 180 mg/mL, about 4 mg/mL to about 170 mg/mL, about 5 mg/mL to about 160 mg/mL, about 6 mg/mL to about 150 mg/mL, about 7 mg/mL to about 130 mg/mL, about 8 mg/mL to about 100 mg/mL, about 9 mg/mL to about 70 mg/mL, from about 10 mg/mL to about 50 mg/mL, or about 15 mg/mL to about 25 mg/mL. The resulting suspensions can then be dispensed into the pellet-forming receptacle for cryopreservation.

Cells can be frozen within about 1 hour, about 2 hours, about 4 hours, 6 hours, about 9 hours, about 12 hours, about 15 hours, about 20 hours, about 24 hours, about 28 hours, about 32 hours, or about 36 hours after cells/tissues/organ harvesting. However, a longer or shorter period of time between the isolation of cells and the subsequent cryopreservation is possible, depending upon the cell preparation desired. For example, cells can be cryopreserved immediately after isolation, or as soon as reasonably possible after isolation (i.e., within 1 hour or less). Alternatively, cells may be cryopreserved after about 48 hours or longer after the isolation.

### Implant

Disclosed herein are implants for treating a skin condition. An implant (e.g., transplant) can be a device, material, or tissue inserted or placed, permanently or temporarily, into or onto a subject (e.g., recipient). An implant can also be used for the administration or delivery of a therapeutic agent. An implant can comprise any of the compositions and formulations disclosed herein.

The implant can be an autologous implant. For example, the implant is derived from the same individual as the recipient. The implant can be an allogeneic implant. For example, the implant is derived from a separate individual of the same species. The implant can be an xenogeneic implant. For example, the implant is derived from an individual of a different species.

The implant can comprise a tissue sample. The tissue sample can be derived from an adipose tissue, an amnion tissue, an artery tissue, a bone tissue, a cartilage tissue, a chorion tissue, a colon tissue, a dental tissue, a dermal tissue, a duodenum tissue, an epithelial tissue, a fascial tissue, a gastrointestinal tissue, a growth plate tissue, an intestinal mucosal tissue, an intestinal serosal tissue, an intervertebral disc tissue, a kidney tissue, a ligament tissue, a liver tissue, a lung tissue, a mammary tissue, a meniscal tissue, a muscle tissue, a nerve tissue, an ovary tissue, a pancreas tissue, a parenchymal organ tissue, a pericardial tissue, a periosteal tissue, a peritoneal tissue, a placental tissue, a reproductive epithelial tissue, a respiratory epithelial tissue, a skin tissue, a spleen tissue, a stomach tissue, a synovial tissue, a tendon tissue, a testes tissue, an umbilical cord tissue, a urological tissue, a vascular tissue, a vein tissue, or, a combination thereof.

### Kits

Disclosed herein are kits for treating a skin condition. Kits useful in the methods of the disclosure comprise components useful in any of the compositions, formulations and methods described herein. The kits can for example, include necessary buffers and/or reagents for treating a skin condition. In some cases, a kit can be a pharmaceutical pack. The kit can comprise a therapeutic agent suitable for treating a skin condition and a set of instructions for administration of the therapeutic agent to a subject in need thereof. In some cases, a kit can also comprise a therapeutic agent suitable for treating a skin condition and a packaging material that contains the therapeutic agent.

### Methods

Disclosed herein are methods of administering the composition to a subject, for example, a human subject in need thereof. The subject can have or be suspected of having a condition. In some embodiments, the condition comprises a skin condition, such as a wound.

The method can promoting the closure of a wound in a patient using the composition disclosed herein. For example, the method can be useful for clinical and personal wound care and soft tissue regeneration. In accordance with the method, the composition can be applied to the wound.

The method can promote closure of external (e.g., surface) and/or internal wounds. The method may be employed alone or as an adjunct to other methods for healing wounded tissue.

The composition can be applied directly to a wound or treatment site of a subject. As described elsewhere herein, the composition may be incorporated into a pharmaceutical formulation including topical ointments, creams, aerosol sprays, and the like.

The method comprises using a biocompatible scaffold as a wound dressing or graft for the skin condition (e.g., wound). The scaffold may be used to treat wounds resulting from trauma, burns, ulcers, abrasions, lacerations, surgery, or other damage. Surgeons can use these scaffolds to cover and protect the wound area, to temporarily replace lost or damaged skin tissue, and/or to guide new tissue generation and wound healing into the damaged area. The scaffold may be secured to the wound area using sutures, adhesives, or overlaying bandages. The scaffold may be cut to match the size of the wound, or may overlap the wound edges. The scaffold may be shaped to penetrate into cavities formed by deep wounds.

The method can comprise using the compositions for personal and home care. The composition can be applied to bandage. The composition can be combined with an adhesive backing to create a bandage. An adhesive section may hold the composition in place on a wounded area and may be removed when the composition degrades or fuse with the tissue. The composition may also be secured with a liquid or gel adhesive. Compositions may also be used as gauze to absorb fluid and/or protect large wounds. This gauze may be wrapped around a wounded area or secured with tape.

The method may be used to treat internal soft tissue wounds such as wounds in the amniotic sac, ulcers in the gastrointestinal tract or mucous membranes, gingival damage or recession, internal surgical incisions or biopsies, etc. The method can comprise suturing or adhering the composition to fill or cover the damaged tissue area.

### Process

Disclosed herein are processes for forming a composition, a formulation, or a kit. The process can comprise forming a composition, a formulation, or a kit for treating a skin condition.

### Isolating Cells

Cells may be isolated from a number of sources, including, for example, from umbilical cord tissue. The isolated cells can be autologous cells, obtained by biopsy from the subject intended to be the recipient. The isolated cells can be allogenic cells, obtained from a subject that is different individual but within the same species as the recipient.

Cells may be isolated using techniques known to those skilled in the art. For example, the tissue or organ may be disaggregated mechanically and/or treated with digestive enzymes and/or chelating agents that weaken the connections between neighboring cells making it possible to disperse the tissue into a suspension of individual cells without appreciable cell breakage. Enzymatic dissociation may be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase and dispase. Mechanical disruption may also be accomplished by a number of methods including, but not limited to, scraping the surface of the organ, the use of grinders, blenders, sieves, homogenizers, pressure cells, or in sonicators. Preferred cell types include stem cells, such as mesenchymal stem cells (MSCs). The preferred cells can be MSCs isolated from umbilical cord tissue.

Once the tissue has been reduced to a suspension of individual cells, the suspension may be fractionated into subpopulations from which the cells elements may be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting.

Cell fractionation may also be desirable, for example, when the donor has diseases such as cancer or metastasis of other tumors to the desired tissue. A cell population may be sorted to separate malignant cells or other tumor cells from normal noncancerous cells. The normal noncancerous cells, isolated from one or more sorting techniques, may then be used for organ reconstruction.

The process can isolate at least one component of the composition. For example, the process can isolate at least one component of a plurality of isolated stem cells, an isolated inductive component, and an isolated scaffolding component. In some cases, the process can isolate at least one of stem cells, Wharton's jelly, and birthing tissue. In some cases, the process can combine two or more of the isolated components.

### Processing Cells

Isolated cells may be cultured in vitro to increase the number of cells available for the process and/or for coating the biocompatible scaffold. If an immunological response occurs in the subject after implantation of the artificial organ, the subject may be treated with immunosuppressive agents such as, cyclosporin or FK506, to reduce the likelihood of rejection. In certain embodiments, chimeric cells, or cells from a transgenic animal, may be used in the process and/or coated onto the biocompatible scaffold.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chance of rejection by the host. In addition, transfection could also be used for gene delivery.

Isolated cells can be expanded ex vivo. The cells can be expanded ex vivo prior to forming the composition. The cells can be expanded ex vivo after forming the composition.

Isolated cells may be normal or genetically engineered to provide additional or normal function. Methods for genetically engineering cells with retroviral vectors, polyethylene glycol, or other methods known to those skilled in the art may be used. These include using expression vectors which transport and express nucleic acid molecules in the cells. (See Goeddel; Gene Expression Technology: Methods in Enzymology 185, Academic Press, San Diego, Calif. (1990).

Vector DNA is introduced into prokaryotic or cells via conventional transformation or transfection techniques. Suitable methods for transforming or transfecting host cells can be found in Sambrook et al. (Molecular Cloning: A Laboratory Manual, 3nd Edition, Cold Spring Harbor Laboratory press (2001)), and other laboratory textbooks.

The process can comprise a cryopreservation process. The process can comprise a cryopreservation process where organelles, cells, tissues, extracellular matrix, organs or any other biological samples are preserved by cooling to a low temperature. For example, the cryopreservation can be carried out at −80° C. using solid carbon dioxide or at −196° C. using liquid nitrogen. The cryopreservation process can protect biological tissue from freezing damage due to ice formation.

The cryopreservation can be carried out by contacting the material to be frozen with a cryoprotectant. In some cases, the cryoprotectant can be glycols (e.g., alcohols containing at least two hydroxyl groups). For example, the cryoprotectant can be ethylene glycol, propylene glycol, or glycerol. In some cases, the cryoprotectant can be Dimethyl sulfoxide (DMSO). In some cases, the cryoprotectant can be a sugar. For example, the cryoprotectant can be sucrose or trehalose. In some cases, the cryoprotectant can be glucose. In some cases, the cryoprotectant can be a polyol. For example, the cryoprotectant can be maltitol, sorbitol, xylitol, erythritol, or isomalt. In some cases, the cryopreserved composition may not include stem cells.

The process can comprise a lyophilization process. The lyophilization process can be a freeze-drying process. The lyophilization process can be a dehydration process that freezes the material and then reduces the surrounding pressure to allow the frozen water in the material to sublime directly from the solid phase to the gas phase. The lyophilization process can comprise a pretreatment, freezing, primary drying, or secondary drying step. The pretreatment step can comprise concentrating the product, formulation revision, decreasing a high-vapor-pressure solvent, or increasing the surface area. The freezing step can comprise placing the material in a freeze-drying flask and/or rotating the flask in a bath, which can be cooled by mechanical refrigeration, dry ice in aqueous methanol, liquid nitrogen, or any combinations thereof. In some cases, the freezing temperatures can be from about −50° C. to −80° C. The primary drying phase can comprise lowing the pressure (e.g., using a partial vacuum) and/or suppling heat to the material. The secondary drying phase can comprise removing unfrozen water molecules. In some cases, the lyophilized composition may not include stem cells.

The cryopreserved and/or lyophilized composition can be stable for at least about 1 hour, for example, at least about 1 hour, about 2 hours, 3 hours, 4 hours, 5 hours, 6 hours, 12 hours, about 24 hours, 36 hours, 48 hours, 3 days, 4, days, 5 days, 6 days, 7 days, about 2 weeks, 3 weeks, 4 weeks, 1 month, about 2 months, 3 months, 4 months, 5 months, 6 months, 1 year, about 2 years, 3 years, 4 years, or 5 years. In some cases, the stability of the composition can be determined by carrying out an activity assay at a pre-determined temperature, for example, at room temperature, at 4° C., at −78.5° C., or at −196° C. In some cases, the activity assay is an assay quantifying the therapeutic activity of the composition.

### Forming Matrices or Scaffolds

In some embodiments, the process can be a process of forming matrices or scaffolds for treating a skin condition. A biocompatible scaffold may be shaped using methods such as, for example, solvent casting, compression molding, filament drawing, meshing, leaching, weaving, foaming, electrospinning and coating.

In solvent casting, a solution of one or more proteins in an appropriate solvent, can be casted as a branching pattern relief structure. After solvent evaporation, a thin film can be obtained. In compression molding, a polymer can be pressed at pressures up to 30,000 pounds per square inch into an appropriate pattern. Filament drawing can involve drawing from the molten polymer and meshing involves forming a mesh by compressing fibers into a felt-like material. In leaching, a solution containing two materials can be spread into a shape close to the final form of the artificial organ. Next a solvent can be used to dissolve away one of the components, resulting in pore formation.

The scaffold may be shaped into any number of desirable configurations to satisfy any number of overall system, geometry or space restrictions. For example, in the use of the scaffold for bladder, urethra, valve, or blood vessel reconstruction, the matrix or scaffold may be shaped to conform to the dimensions and shapes of the whole or a part of the tissue. The scaffold may be shaped in different sizes and shapes to conform to the organs of differently sized patients. For bladders, the scaffold should be shaped such that after its biodegradation, the resulting reconstructed bladder may be collapsible when empty in a fashion similar to a natural bladder. The matrix or scaffold may also be shaped in other fashions to accommodate the special needs of the patient.

In one embodiment, the scaffolds are seeded with one or more populations of cells to form an artificial organ construct. The artificial organ construct may be autologous, where the cell populations are derived from the subject's own tissue, or allogenic, where the cell populations are derived from another subject within the same species as the patient. The artificial organ construct may also be xenogenic, where the different cell populations are derived form a mammalian species that is different from the subject. For example the cells may be derived from organs of mammals such as humans, monkeys, dogs, cats, mice, rats, cows, horses, pigs, goats and sheep.

Seeding of cells onto the matrix or scaffold may be performed according to standard methods. For example, the seeding of cells onto polymeric substrates for use in tissue repair has been reported (see, e.g., Atala, A. et al., J. Urol. 148(2 Pt 2): 658-62 (1992); Atala, A., et al. J. Urol. 150 (2 Pt 2): 608-12 (1993)). Cells grown in culture may be trypsinized to separate the cells, and the separated cells may be seeded on the matrix. Alternatively, cells obtained from cell culture may be lifted from a culture plate as a cell layer, and the cell layer may be directly seeded onto the scaffold without prior separation of the cells.

### Making a Formulation

The process can make a formulation for treating a skin condition. The formulation can have from about 1 million to about 50 million cells (e.g., MSC cells). The cells can be incubated under standard culturing conditions, such as, for example, 37° C., 5% CO₂, for a period of time. Greater cell densities can promote greater tissue regeneration by the seeded cells, while lesser densities can permit relatively greater regeneration of tissue by cells infiltrating the graft from the host. Other seeding techniques may also be used depending on the matrix or scaffold and the cells. For example, the cells may be applied to the matrix or scaffold by vacuum filtration.

In order to facilitate cell growth on the skin, the scaffold may be coated with one or more cell adhesion-enhancing agents. These agents include but are not limited collagen, laminin, and fibronectin. The scaffold may also contain cells cultured on the scaffold to form a target tissue substitute. The target tissue that may be formed using the scaffold of the present invention may be an arterial blood vessel, wherein an array of microfibers is arranged to mimic the configuration of elastin in the medial layer of an arterial blood vessel. In the alternative, other cells may be cultured on the scaffold of the present invention. These cells include, but are not limited to, cells cultured on the scaffold to form a blood vessel substitute, epithelial cells cultured on the scaffold to form epithelial tissue, muscle cells cultured on the scaffold to form muscle tissue, endothelial cells cultured on the scaffold to form endothelial tissue, skeletal muscle cells cultured on the scaffold to form skeletal muscle tissue, cardiac muscle cells cultured on the scaffold to form cardiac muscle tissue, collagen fibers cultured on the scaffold to form cartilage, interstitial valvular cells cultured on the scaffold to form valvular tissue and mixtures thereof.

Methods for coating a material with a chemical composition include, but are not limited to, methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The compositions described herein can have the ability to fill irregular and deep wounds. The composition can be a pharmaceutical composition. The composition can be topically applied to a wound or to a site in need of tissue regeneration.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials can increase the rate of penetration of drugs across the skin. Typical enhancers can include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, and N-methyl-2-pyrrolidone.

The formulations may be combined with other ingredients such as adjuvants, anti-oxidants, chelating agents, surfactants, foaming agents, wetting agents, emulsifying agents, viscosifiers, buffering agents, preservatives, or the like. A permeation or penetration enhancer can be included in the composition and/or can be effective in improving the percutaneous penetration of the composition with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, and N-methyl-2-pyrrolidone, can be used in the process. The formulation may further comprise a hydrotropic agent, such as isopropyl alcohol, propylene glycol, or sodium xylene sulfonate.

The formulations can be applied in an amount effective to affect desired changes. As used herein “amount effective” shall mean an amount sufficient to cover the region of skin surface where a change is desired. An active compound should be present in the amount of from about 0.0001% to about 50% by weight volume of the composition.

The formulations can be applied to a bandage or dressing, which is then applied to the wound or treatment site of a subject. For example, a dressing can be soaked in a liquid solution or liquid suspension comprising the composition. In another embodiment, an ointment comprising the composition can be applied to a surface of a dressing or bandage.

### EXAMPLE

### Example 1—Method of Preparing an Umbilical Cord Sample

A human umbilical cord (HUC) sample is obtained from a normal birth. Umbilical cord blood is collected from the umbilical cord sample. Mesenchymal stem cells (MSCs) are isolated from the umbilical cord blood.

Umbilical cord veins are removed with a vein stripper or by longitudinally cutting the umbilical cord to expose the veins for removal. The umbilical cord membrane and Wharton's jelly are isolated and cut into sections of approximately 10 cm to 15 cm in length.

The umbilical cord is then rinsed in cold 0.9% saline, and placed in a 0.9% phosphate-buffered saline solution containing 10 mM EDTA and 10 mM EGTA, at a ratio of about 100 ml solution for every gram of tissue, at 4° C. The solution is acidified to about pH 4.5 using glacial acetic acid. The pH of the solution is determined using a standard pH meter. The solution and umbilical cord is placed into a blender, and the umbilical cord is homogenized for one minute at the highest setting to produce an umbilical cord suspension. The acidified suspension is allowed to stand at 4° C., for 24 hours. After 24 hours, the suspension is brought to about pH 7.4 with 10N NaOH, and divided into four culture bottles suitable for use in a swinging bucket centrifuge rotor. The suspension is centrifuged at 4° C. for 10 minutes at 5000 g to produce a supernatant. The supernatant is optionally centrifuged again at 4° C. and 10000 g. The supernatant is dialyzed over a semipermeable membrane against 20 volumes of deionized water, and stored for further use.

The samples containing MSCs, Wharton's jelly and the umbilical cord membrane are mixed with a viscosity modifying component, such as alginic acid or glycerol gel, to generate the final composition. The viscosity modifying component is from about 1% to about 20% by weight of the composition. Optionally, the composition is dyed and processed by a grinder to make a powder formulation.

### Example 2—Method of Preparing a Solution

The umbilical cord is separated from the placental disc. Umbilical cord blood is collected from the umbilical cord sample. Mesenchymal stem cells (MSCs) are isolated from the umbilical cord blood.

The umbilical cord is rinsed in a sterile saline solution and sectioned into pieces of about 10 cm to about 15 cm in length. The umbilical cord is then slit or cut longitudinally using a pair of surgical scissors so that the two arteries and one vein are exposed. The vessels are then removed with a forceps. The umbilical cord membrane, including Wharton's jelly, is then rinsed in cold saline or other balanced salt solution at neutral pH. The solution optionally comprises one or more antibiotics. The umbilical cord membrane can be further processed immediately, or can be stored in a refrigerator for up to about 72 hours in a sterile buffered saline solution, such as 0.9% sterile NaCl solution.

Homogenization of HUC at neutral pH. The following procedures are all carried out at 4° C. and neutral pH (approximately 7.4) under sterile conditions. The umbilical cord membrane is cut into small pieces and weighed. Sterile buffer, and optionally antibiotics, are then added to the chamber of a homogenizer to reach the desired weight to volume ratio (w/v) of 1:5. The membrane pieces are then homogenized to produce an umbilical cord membrane suspension. Homogenization is repeated as necessary in order to obtain a visibly homogenous suspension without raising the temperature above about 4° C. The resulting suspension can be used without further treatment.

The homogenate is centrifuged for 10 minutes at 4° C. and 3500 rpm and then the supernatant is further centrifuged for another 5 minutes at 10000 rpm to get rid of any undesired particulate matter present in the extract to produce an umbilical cord solution. The obtained umbilical cord membrane solution is analyzed by a Lowry assay to quantify the total protein amount present in the homogenate. The solution is then filtered through 0.45 micron filters under a sterile hood and stored at 4° C. The solution is then mixed with an alginic acid solution to generate the final solution.

### Example 3—Method of Preparing a Paste

A solution is obtained in the same manner described above in Example 2. The resulting solution is dehydrated using a vacuum dehydration apparatus at about 4° C. Dehydration is continued until the water in the solution is substantially removed and the remaining umbilical cord material appears dry by visual inspection. The mass of the umbilical cord material is determined, and water or buffer is then added to the umbilical cord material to achieve a water content of about 3% to about 10% by weight. The water and umbilical cord material are thoroughly mixed to form a paste.

The umbilical cord material is supplemented by an amount of purified collagen not to exceed 100 times the weight of the umbilical cord material obtained from the umbilical cord solution, and water is added to as to achieve between 3% and 10% water by weight. The water, umbilical cord material and purified collagen are thoroughly mixed to form a paste.

### Example 4—Method of Preparing a Gel

A solution is obtained in the same manner described above in Example 2. The resulting solution is dehydrated using a vacuum dehydration apparatus at about 4° C. Dehydration is continued until the water in the solution is substantially removed and the remaining umbilical cord material appears dry by visual inspection. The mass of the umbilical cord material is determined, and a gelling compound is added in a weight/weight ratio to the umbilical cord material of about 10:1. Water is then added to the umbilical cord material and gelling compound in an amount that brings the water content of the resulting gel to about 60% to about 99% by weight.

In a variation of the above method, the umbilical cord material is supplemented with purified collagen in a weight not to exceed 100 times the weight of the umbilical cord material obtained from the umbilical cord solution. Water is added to the umbilical cord material, purified collagen, and gelling agent to bring, the water content of the resulting gel to 60% to about 99% by weight.

### Example 5—Method of Treating a Wound Using Umbilical Vein Endothelial Cells (HUVEC)

To evaluate the impact on cell migration and wound healing, the above described compositions are tested with human umbilical vein endothelial cells, which are derived from the endothelium of veins from the umbilical cord. HUVEC have been widely used as a model system for the study of the regulation of endothelial cell function and the role of the endothelium in response of the blood vessel wall to stretch, shear forces and the development of atherosclerotic plaques and angiogenesis.

HUVEC cells are pre-treated with or without 30 mM glucose for 3 days (72 hours). The cells are then equilibrated for a day (24 hours), and incubated with the compositions described in the above examples for 24 hours before wounding.

Images are taken at intervals and the wound gap is quantified over time using Image analysis software. For example, an initial image is taken at the time of injury (i.e., scratch) and the distance is measured; thereafter images are taken over incremental time periods and distances are measured at 24 hours. 100 measurements are taken per image and a mean gap distance is determined. The time for 50% of the wound to heal is quantified as well as the percentage of improvement compared to untreated cells.

### Example 6—Method of Treating a Wound and Evaluate Formulations

The precise ranges of each of the components utilized within the compositions are evaluated. The effects of the individual components of the claimed compositions on the growth of a vascularized endothelial cell line (e.g. cells of the stomach lining) are tested and determined as controls.

To calculate the needed ratios for the claimed compositions, a combination dosage study is conducted using the same testing method as described above on every two-ingredient combinations for all the ingredients. Ingredient concentrations is determined using the top two preforming concentrations from the initial study. Further component amount/ratio evaluation is continued. Ingredient synergy is observed for some combinations.

### Example 7—Animal Models for Wound Treatment

Wounds, about 2.0×2.0 cm full thickness, are created on the dorsal skin of nude mice. The above described compositions are deposited and crosslinked within the wounds. Control mice receive no treatment other than standard bandaging procedures. Wound size, re-epithelialization, and contraction are measured immediately after surgery, and at day 4, 7, 10, and 14, after which the animals are euthanized and the regenerated skin is harvested for histological and biochemical analysis.

In the porcine model, full thickness 4.0×4.0 cm full thickness wounds are created on the dorsal flanks of Specific Pathogen Free (SPF) Yorkshire pigs. The above described compositions are deposited and crosslinked within the wounds. Additionally, sterilized powder, is applied to the wounds, either directly as a powder, or following resuspension in saline solution. For comparisons, some control wounds receive other commercially available wound healing products. Additional control wounds receive no treatment other than standard bandaging procedures. Wound size, re-epithelialization, and contraction are measured immediately after surgery, and at day 4, 7, 10, 14, 18, about 21, about 24, and 28 after which the animals are euthanized and the regenerated skin is harvested for histological and biochemical analysis.

### Example 8—Method of Treating a Wound Using Animal Model

An in vivo wound healing study is performed to evaluate the efficacy of the compositions described herein. A 2 cm by 2 cm full-thickness wound is created on the back of nude mice. Each wounded mouse received one of 3 treatment options; (1) Untreated other than standard bandage, (2) a commercially available wound healing product only, or (3) a composition described herein (e.g., powder, solution, gel, and paste). Time-course images of the wounds are recorded. Gross morphology of the wounds demonstrates accelerated wound closure times. Percentage (%) wound remaining is calculated by dividing the area of the remaining wound by the original wound size. Wound re-epithelialization is calculated by measuring newly re-epithelized skin, taking into consideration remaining wound area and contraction. Contracture is measured based on original wound size and the area of re-epithelized skin. Wound aspect ratio is determined to describe observed changes in the shape and direction of wound contraction between groups. Similar study is also performed in the porcine model.

Wound area sections are subjected to hematoxylin and eoasin stain (H&E stain) to detect blood vessels within the tissue. Blood vessel density within areas of regenerated skin is quantified by counting performed on multiple representative fields of view, which demonstrates increased density for treated animals compared to both untreated and commercial product only groups. The blood vessel area is calculated and represented as relative to blood vessel size within healthy skin of the same mouse. The distribution of blood vessels between large, medium and small vessels is determined. Significantly more smaller blood vessels suggests new blood vessel formation.

Blood vessels in regenerating skin are subjected to immuno-fluorescent staining for a-smooth muscle actin (aSMA) and von willebrand factor (vWF). The epidermis of the animals are subject to immuno-fluorescent staining for keratin 10, to detect epidermis, and Ki-67, to detect proliferating cells. The skin thickness and cell proliferation are also examined in treated animals, compared to untreated skin.

Experiments are conducted to evaluate protein release from the treatment. Protein release from the treatment sites is observed over a 14 day time period demonstrating the controlled and extended release of factors over time. Kinetic models of protein release are fit to the observed cumulative protein release to investigate the mechanism of protein release.

Wounds untreated (control), or treated with commercial product or the disclosed compositions are subjected to histochemical staining of various stains. Herovici's Collagen Stain, Massons Trichrome and Picrosirius Red staining illustrate modified collagen types, maturity and distribution compared to untreated skin. Alcian Blue and Verhoeff van Geison staining also reveal differences in elastin, proteoglycan and glycosaminoglycan composition between the groups.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
(a) an isolated and processed birthing tissue comprising an umbilical cord tissue, wherein said birthing tissue comprises from about 10% to about 95% by weight of said composition, and
(b) at least two of:
(i) a plurality of isolated stem cells,
(ii) an isolated inductive component comprising a portion of a Wharton's jelly,
(iii) a viscosity modifying component, and
(iv) a cryopreservative.

2. The composition of claim 1, comprising said viscosity modifying component, wherein said viscosity modifying component comprises a polyol, a sugar, a derivative of any of these, a salt of any of these, or any combination thereof.

3. The composition of claim 1, comprising said viscosity modifying component, wherein said viscosity modifying component comprises a polyol, a derivative thereof, a salt of any of these, or any combination thereof.

4. The composition of claim 3, wherein said polyol, said derivative thereof, or said salt of any of these is a polyhydric alcohol, a derivative thereof, or a salt of any of these.

5. The composition of claim 4, comprising said polyhydric alcohol, said derivative thereof, or said salt of any of these, wherein said polyhydric alcohol, said derivative thereof, or said salt of any of these is a glycerol, an erythritol, a threitol, an arabitol, a xylitol, a ribitol, a mannitol, a sorbitol, a galactitol, a fucitol, an iditol, an inositol, a volemitol, an isomalt, a maltitol, a lactitol, a maltotriitol, a maltotetraitol, a polyglycitol, a salt of any of these, a derivative of any of these, or any combination thereof.

6. The composition of claim 5, comprising said salt of any of these or said derivative of any of these, and wherein said salt of any of these or said derivative of any of these is alkylated, acetylated, phosphorylated, ubiquitinated, glycosylated, methylated, hydroxylated, sulfated, sulfonated, pegylated, propylene glycolated, contains an amino group, contains an imino group, or any combination thereof.

7. The composition of claim 4, wherein said polyhydric alcohol or said salt thereof is a C₃-C₃₀ polyhydric alcohol, a derivative thereof, or a salt of any of these.

8. The composition of any claim, comprising said viscosity modifying component, wherein said viscosity modifying component comprises a polymer.

9. The composition of claim 8, wherein said polymer comprises a plurality of sugar monomers.

10. The composition of claim 8, wherein said polymer comprises an alginic acid or a salt thereof.

11. The composition of any claim, comprising said viscosity modifying component, wherein said viscosity modifying component comprises a carboxylic acid or a salt thereof.

12. The composition of any claim, comprising said viscosity modifying component, wherein said viscosity modifying component comprises a gelatin or a derivative thereof.

13. The composition of any claim, comprising said viscosity modifying component, wherein said viscosity modifying component comprises collagen.

14. The composition of claim 13, wherein said collagen is at least partially hydrolyzed.

15. The composition of any claim comprising said viscosity modifying component, wherein said viscosity modifying component is selected from the group consisting of: carboxylic acid copolymers, carboxylate copolymers, acrylate homopolymers, acrylate copolymers, and any combination thereof.

16. The composition of any claim, comprising said viscosity modifying component, wherein said viscosity modifying component is from about 1% to about 20% by weight of said composition.

17. The composition of claim 16, wherein said viscosity modifying component is from about 12% to about 17% by weight of said composition.

18. The composition of any claim, comprising said plurality of isolated stem cells.

19. The composition of claim 18, wherein said plurality of isolated stem cells comprises mesenchymal stem cells.

20. The composition of claim 18, wherein said plurality of isolated stem cells comprise an autologous stem cell.

21. The composition of claim 18, wherein said plurality of isolated stem cells comprise a heterologous stem cell.

22. The composition of claim 18, wherein at least a portion of said plurality of stem cells is viable.

23. The composition of claim 22, wherein said at least a portion is at least about 80% of a total cell number of said composition.

24. The composition of claim 18, wherein said plurality of isolated stem cells is from about 0.1% to about 5% by weight of said composition.

25. The composition of claim 24, wherein said plurality of isolated stem cells is from about 0.5% to about 2% by weight of said composition.

26. The composition of any claim, comprising said isolated inductive component.

27. The composition of claim 1, comprising said isolated inductive component, wherein said isolated inductive component comprises a mucous tissue.

28. The composition of claim 1, comprising said isolated inductive component, wherein said isolated inductive component comprises a mucopolysaccharide or a salt thereof.

29. The composition of claim 1, comprising said isolated inductive component, wherein said isolated inductive component comprises hyaluronic acid or a salt thereof.

30. The composition of claim 1, comprising said isolated inductive component, wherein said isolated inductive component comprises chondroitin or a salt thereof.

31. The composition of claim 1, comprising said isolated inductive component, wherein said isolated inductive component comprises a salt of chondroitin.

32. The composition of claim 1, comprising said isolated inductive component, wherein said isolated inductive component comprises N-acetylgalactoasmine or a salt thereof.

33. The composition of claim 1, comprising said isolated inductive component, wherein said isolated inductive component comprises glucuronic acid or a salt thereof.

34. The composition of claim 1, comprising said isolated inductive component, wherein said isolated inductive component comprises water.

35. The composition of claim 1, comprising said isolated inductive component, wherein said isolated inductive component comprises a plurality of isolated fibroblasts.

36. The composition of claim 35, wherein at least a portion of said plurality of isolated fibroblasts is viable.

37. The composition of claim 1, comprising said isolated inductive component, wherein said isolated inductive component comprises a plurality of isolated macrophages.

38. The composition of claim 37, wherein at least a portion of said plurality of isolated macrophages is viable.

39. The composition of claim 1, comprising said isolated inductive component, wherein said isolated inductive component is from about 0.1% to about 50% by weight of said composition.

40. The composition of claim 39, wherein said isolated inductive component is from about 30% to about 50% by weight of said composition.

41. The composition of claim 1, comprising said isolated and processed birthing tissue, wherein said isolated and processed birthing tissue further comprises a placental tissue.

42. The composition of claim 1, comprising said isolated and processed birthing tissue, wherein said isolated and processed birthing tissue comprises a conductive property.

43. The composition of claim 1, comprising said isolated and processed birthing tissue, wherein said isolated and processed birthing tissue comprises minced tissue.

44. The composition of claim 1, comprising said isolated and processed birthing tissue, wherein said isolated and processed birthing tissue comprises micronized tissue.

45. The composition of claim 1, comprising said isolated and processed birthing tissue, wherein said isolated and processed birthing tissue comprises lyophilized tissue.

46. The composition of claim 1, comprising said isolated and processed birthing tissue, wherein said isolated and processed birthing tissue comprises a plurality of pieces of tissue.

47. The composition of claim 1, comprising said isolated and processed birthing tissue, wherein said isolated and processed birthing tissue is from about 20% to about 50% by weight of said composition.

48. The composition of claim 47, wherein said isolated and processed birthing tissue is from about 30% to about 50% by weight of said composition.

49. The composition of any claim, further comprising a plurality of epithelial cells.

50. The composition of any claim, further comprising a plurality of endothelial cells.

51. The composition of any claim, further comprising a plurality of perivascular cells.

52. The composition of any claim, further comprising a peptide.

53. The composition of any claim, further comprising a protein.

54. The composition of any claim, further comprising an amino acid.

55. The composition of any claim, further comprising water.

56. The composition of any claim, further comprising at least one growth factor.

57. The composition of claim 56, wherein said at least one growth factor comprises insulin-like growth factor-1, insulin-like growth factor binding protein-3, vascular endothelial growth factor (VEGF), hepatocyte growth factor (HGF), placenta growth factor (PIGF), or any combination thereof.

58. The composition of any claim, further comprising at least one of: chemokine ligand 2, macrophage inflammatory protein-1 (MIP-1) alpha, MIP-1 beta, MIP-2, beta-chemokine ligand-5, beta-chemokine ligand-20, alpha-chemokine ligand-14, lipopolysaccharide-induced alpha-chemokine, Granulocyte-macrophage colony-stimulating factor, interleukin IL-1 beta, phorbol myristate acetate, epidermal growth factor, fibroblast growth factor, vascular endothelial growth factor, connective tissue growth factor, platelet-derived growth factor, insulin-like growth factor, nerve growth factor, hepatocyte growth factor, colony-stimulating factor, stem cell factor, keratinocyte growth factor, granulocyte colony-stimulating factor, granulocyte macrophage colony-stimulating factor, glial derived neurotrophic factor, ciliary neurotrophic factor, endothelial-monocyte activating polypeptide, epithelial neutrophil activating peptide, erythropoietin, bone morphogenetic protein, brain-derived neurotrophic factor, transforming growth factor beta, tumor necrosis factor, or any combination thereof.

59. The composition of any claim, further comprising at least one hormone.

60. The composition of claim 59, wherein said at least one hormone is prolactin or leptin.

61. The composition of claim 1, comprising said cryopreservative.

62. The composition of claim 61, wherein said cryopreservative comprises a sucrose, a trehalose, a starch, a salt of any of these, a derivative of any of these, or any combination thereof.

63. The composition of any claim, which is in unit dose form.

64. The composition of any claim, which is formulated as a liquid, an aerosol, an aerosolized liquid, a foam, a cream, a gel, an ointment, a putty, a semi-solid, a glue, a coating, an adhesive, or any combination thereof.

65. The composition of any claim, comprising a viscosity from about 0.5 centipoise (cp) to about 2 cp at 70° F.

66. The composition of any claim, comprising a viscosity from about 100 cp to about 900 cp at 70° F.

67. The composition of any claim, comprising a viscosity from about 1,000 cp to about 5,000 cp at 70° F.

68. The composition of any claim, comprising a viscosity from about 50,000 cp to about 80,000 cp at 70° F.

69. The composition of any claim, comprising a viscosity from about 90,000 cp to about 150,000 cp at 70° F.

70. The composition of any claim, comprising a viscosity from about 200,000 cp to about 300,000 cp at 70° F.

71. The composition of any claim, wherein said composition maintains at least about 90% of a wound healing activity after storage in a container at an average temperature from about 25 degrees Celsius to about 200 degrees Celsius.

72. The composition of claim 71, wherein said container is a sealed container.

73. A method comprising: administering said composition of any preceding claim to a subject.

74. The method of claim 73, wherein said subject is a subject in need thereof.

75. The method of claim 73, wherein said subject is a human subject.

76. The method of claim 73, wherein said subject is a subject having or suspected of having a condition.

77. The method of claim 76, wherein said condition comprises a skin condition.

78. The method of claim 76, wherein a time of healing from said condition for said subject is reduced by at least about 10% when compared to another time of healing for a comparable condition for another subject, wherein said comparable condition of said another subject is not treated with said composition.

79. The method of claim 73, wherein said administering comprises contacting said composition to an abrasion, an incision, a peel, a laceration, a puncture, an avulsion, a contusion, an amputation, a chemical wound, a thermal wound, an electrical wound, an ulcer, a sore, a hematoma, a burn, or any combination thereof.

80. A process comprising: forming a composition by combining
(a) an isolated and processed birthing tissue comprising an umbilical cord tissue, wherein said birthing tissue comprises from about 10% to about 95% by weight of said composition, and
(b) at least two of:
(i) a plurality of isolated stem cells,
(ii) an isolated inductive component comprising a portion of a Wharton's jelly, and
(iii) a viscosity modifying component, and
(iv) a cryopreservative.

81. The process of claim 80, further comprising: adding a preservative, an anti-irritant, or a combination thereof to said composition.

82. The process of claim 80, further comprising: treating said composition with a solution comprising at least one selected from the group consisting of: an antibiotic, an antimycotic, and a combination thereof.
